(19) United States
(12) Patent Application Publication
(10) Pub. No.: US 2020/0276297 A1
(43) Pub. Date: Sep. 3, 2020

(54) VACCINES AND METHODS OF MAKING AND USING VACCINES FOR PREVENTION OF RESPIRATORY SYNCYTIAL VIRUS (RSV) INFECTIONS

(71) Applicants: Ohio State Innovation Foundation, Columbaus, OH (US); THE RESEARCH INSTITUTE AT NATIONWIDE CHILDREN'S HOSPITAL, Collumbus, OH (US)

(72) Inventors: Stefan NIEWIESK, Dublin, OH (US); Basavaraj BINJAWADAGI, Maryland Heights, MO (US); Jianrong LI, Dublin, OH (US); Mark PEEPLES, Bexley, OH (US)

(21) Appl. No.: 16/647,758

(22) PCT Filed: Sep. 14, 2018
(86) PCT No.: PCT/US2018/051054

§ 371 Date: Mar. 16, 2020

Related U.S. Application Data
(60) Provisional application No. 62/559,167, filed on Sep. 15, 2017.

(51) Int. Cl. A61K 39/155; C12N 7/00; A61P 31/14

(52) U.S. Cl. CPC A61K 39/155; A61K 2039/53; A61P 31/14; C12N 7/00

(57) ABSTRACT

Disclosed herein are vaccines, immunogenic compositions, and methods of using the same to treat and prevent respiratory syncytial virus (RSV). Specifically, disclosed are immunogenic compositions wherein a protein or immunogenic fragment of RSV is delivered to a subject in a recombinant viral vector platform, such as vesicular stomatis virus (rVSV).

### CROSS-REFERENCE TO RELATED APPLICATIONS

This application claims benefit of U.S. Provisional Application No. 62/559,167, filed Sep. 15, 2017, which is incorporated herein by reference in its entirety.

### BACKGROUND

A global study has found that RSV is one of the most common causes of infant hospitalization due to acute lower respiratory tract infections (ALRI) in children younger than 5 years of age in the US and worldwide, resulting in up to 200,000 deaths. RSV was associated with hospitalizations 16-times more than influenza in children under one year of age. In addition to hospitalization, RSV resulted in higher rates of emergency department visits and required more caregiver time and resource utilization than influenza.

Currently, several RSV vaccine candidates are under development or clinical trials targeting different age groups. Both live attenuated and killed vaccines have been attempted, but without much success. Recombinant viral vectors, such as recombinant vesicular stomatitis virus (rVSV), adenovirus, etc., provide powerful technologies for delivering heterologous antigens (antigens from different viruses) with minimal disadvantages. What is needed in the art is an efficacious rVSV vector based anti-RSV vaccine that safely used in humans to prevent RSV infections.

### SUMMARY

Disclosed herein are compositions comprising a recombinant viral vector and one or more respiratory syncytial virus (RSV) proteins.

Also disclosed herein are methods of using the immunogenic compositions and vaccines disclosed herein. For example, disclosed are methods of eliciting an immune response against RSV in a subject, the method comprising administering to the subject a composition or vaccine as disclosed herein.

### BRIEF DESCRIPTION OF THE DRAWINGS

FIG. 1 shows a schematic representation of the VSV vector (Indiana strain; sequence listed as the last sequence in the list of sequences) with the location for cloning of the RSV genes.

FIGS. 2A, 2B, and 2C show clearance of challenge virus (a and b) and VN antibody titers (c) in the rVSV-G±F immunized cotton rats. Cotton rats (n=4 per group) were immunized with indicated dose and combination of the rVSV candidates and challenged with RSV-A2 four weeks after immunization and euthanized four days after challenge. Virus titration was done using lung and nasal homogenates collected on the day of euthanization and VN antibody levels were determined from the serum samples collected on the day of challenge. Statistical analysis was done by one-way ANOVA and statistically significant difference (at P<0.05) between indicated group representing bars is indicated by asterisk (*) symbol.

FIGS. 3A, 3B, and 3C show clearance of challenge virus (a and b) and VN antibody titers (c) in the rVSV-G±F immunized cotton rats. Cotton rats (n=4 per group) were immunized with indicated dose, interval and combination of the rVSV candidates and challenged with RSV-A2 three weeks after booster dose and euthanized four days after challenge. Virus titration was done using lung and nasal homogenates and VN antibody levels were determined from the serum samples collected on the day of booster immunization (day 21) and RSV challenge (day 42). Statistical analysis was done by one-way ANOVA and statistically significant difference (at P<0.05) between indicated groups representing bars is indicated by asterisk (*) symbol.

FIGS. 4A, 4B, and 4C show clearance of challenge virus (a and b) and VN antibody titers (c) in the indicated rVSV-G+F+rVSV-Hsp70 immunized cotton rats. Cotton rats (n=4 per group) were immunized with indicated dose, interval and combination of the rVSV candidates and challenged with RSV-A2 three weeks after booster dose and euthanized four days after challenge. Virus titration was done using lung and nasal homogenates and VN antibody levels were determined from the serum samples collected on the day of booster immunization (day 21) and RSV challenge (day 42). Statistical analysis was done by one-way ANOVA and statistically significant difference (at P<0.05) between indicated groups representing bars is indicated by asterisk (*) symbol.

FIGS. 5A, 5B, 5C show clearance of challenge virus (a and b) and VN antibody titers (c) in the indicated variant of RSV G expressing rVSV immunized cotton rats. Cotton rats (n=4 per group) were immunized with indicated dose, interval and combination of the rVSV candidates and challenged with RSV-A2 three weeks after booster dose and euthanized four days after challenge. Virus titration was done using lung and nasal homogenates and VN antibody levels were determined from the serum samples collected on the day of booster immunization (day 21) and RSV challenge (day 42). Statistical analysis was done by one-way ANOVA and statistically significant difference (at P<0.05) between indicated groups representing bars is indicated by asterisk (*) symbol.

FIGS. 6A, 6B, and 6C show clearance of challenge virus (a and b) and VN antibody titers (c) in the rVSV-G variants immunized cotton rats. Cotton rats (n=4 per group) were immunized with indicated dose and combination of the rVSV candidates and challenged with RSV-A2 after four weeks and euthanized four days after challenge. Virus titration was done using lung and nasal homogenates and VN antibody levels were determined from the serum samples collected on the day of challenge. Statistical analysis was done by one-way ANOVA and statistically significant difference (at P<0.05) between indicated group representing bars is indicated by asterisk (*) symbol.

FIG. 7 shows a schematic representation of the ectodomain of the RSV F gene with details of the mutations and substitutions included to stabilize F protein in perfusion conformation (Pre-F).

FIG. 8 shows a schematic representation of RSV N gene and segments of the gene selected for expression in rVSVs vectors as detailed in Table. 3.

### DETAILED DESCRIPTION

The present invention can be understood more readily by reference to the following detailed description of the invention and the Examples included therein.

All patents, patent applications, and publications cited herein, whether supra or infra, are hereby incorporated by reference in their entireties into this application in order to more fully describe the state of the art as known to those skilled therein as of the date of the invention described and claimed herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### Definitions

Unless otherwise defined, all technical and scientific terms used herein have the same meaning as commonly understood by one of ordinary skill in the art to which this disclosure belongs. The terminology used in the description of the embodiments herein is for describing particular embodiments only and is not intended to be limiting of the embodiments disclosed. As used in the description, the singular forms “a,” “an,” and “the” are intended to include the plural forms as well, unless the context clearly indicates otherwise. All publications, patent applications, patents, and other references mentioned herein are incorporated by reference in their entirety.

Unless otherwise indicated, all numbers expressing quantities of ingredients, reaction conditions, and so forth used in this disclosure are to be understood as being modified in all instances by the term “about.” Accordingly, unless indicated to the contrary, the numerical parameters set forth in this disclosure are approximations that may vary depending upon the desired properties sought to be obtained by the present disclosure. At the very least, and not as an attempt to limit the application of the doctrine of equivalents to the scope of any claims, each numerical parameter should be construed in light of the number of significant digits and ordinary rounding approaches.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values described herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data are provided in a number of different formats, and that these data, represent endpoints, starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units is also disclosed. For example, if 10 and 15 are disclosed, then 11, 12, 13, and 14 are also disclosed.

As used herein, the term “amino acid sequence” refers to a list of abbreviations, letters, characters or words representing amino acid residues. The amino acid abbreviations used herein are conventional one letter codes for the amino acids and are expressed as follows: A, alanine; C, cysteine; D aspartic acid; E, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine.

“Polypeptide” as used herein refers to any peptide, oligopeptide, polypeptide, gene product, expression product, or protein. A polypeptide is comprised of consecutive amino acids. The term “polypeptide” encompasses naturally occurring or synthetic molecules. The terms “polypeptide,” “peptide,” and “protein” can be used interchangeably.

In addition, as used herein, the term “polypeptide” refers to amino acids joined to each other by peptide bonds or modified peptide bonds, e.g., peptide isosteres, etc. and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide. Also, a given polypeptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and transfer-RNA mediated addition of amino acids to protein such as arginylation. (See Proteins—Structure and Molecular Properties 2nd Ed., T. E. Creighton, W.H. Freeman and Company, New York (1993); Posttranslational Covalent Modification of Proteins, B. C. Johnson, Ed., Academic Press, New York, pp. 1-12 (1983)).

As used herein, “isolated polypeptide” or “purified polypeptide” is meant to mean a polypeptide (or a fragment thereof) that is substantially free from the materials with which the polypeptide is normally associated in nature. The polypeptides of the invention, or fragments thereof, can be obtained, for example, by extraction from a natural source (for example, a mammalian cell), by expression of a recombinant nucleic acid encoding the polypeptide (for example, in a cell or in a cell-free translation system), or by chemically synthesizing the polypeptide. In addition, polypeptide fragments may be obtained by any of these methods, or by cleaving full length proteins and/or polypeptides.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

As used herein, “isolated nucleic acid” or “purified nucleic acid” is meant to mean DNA that is free of the genes that, in the naturally-occurring genome of the organism from which the DNA of the invention is derived, flank the gene. The term therefore includes, for example, a recombinant DNA which is incorporated into a vector, such as an autonomously replicating plasmid or virus; or incorporated into the genomic DNA of a prokaryote or eukaryote (e.g., a transgene); or which exists as a separate molecule (for example, a cDNA or a genomic or cDNA fragment produced by PCR, restriction endonuclease digestion, or chemical or in vitro synthesis). It also includes a recombinant DNA which is part of a hybrid gene encoding additional polypeptide sequence. The term “isolated nucleic acid” also refers to RNA, e.g., an mRNA molecule that is encoded by an isolated DNA molecule, or that is chemically synthesized, or that is separated or substantially free from at least some cellular components, for example, other types of RNA molecules or polypeptide molecules.

As used herein, “sample” is meant to mean an animal; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample can also be any body fluid or excretion (for example, but not limited to, blood, urine, stool, saliva, tears, bile) that contains cells or cell components.

Although methods and materials similar or equivalent to those described herein can be used in the practice or testing of this disclosure, suitable methods and materials are described below. The term “comprises” means “includes.” Thus, unless the context requires otherwise, the word “comprises,” and variations such as “comprise” and “comprising” will be understood to imply the inclusion of a stated compound or composition (e.g., nucleic acid, polypeptide, antigen) or step, or group of compounds or steps, but not to the exclusion of any other compounds, composition, steps, or groups thereof.

An “immunogenic composition” is a composition of matter suitable for administration to a human or animal subject (e.g., in an experimental setting) that is capable of eliciting a specific immune response, e.g., against a pathogen, such as RSV. As such, an immunogenic composition includes one or more antigens (for example, whole purified virus or antigenic subunits, e.g., polypeptides, thereof) or antigenic epitopes. An immunogenic composition can also include one or more additional components capable of eliciting or enhancing an immune response, such as an excipient, carrier, and/or adjuvant. In certain instances, immunogenic compositions are administered to elicit an immune response that protects the subject against symptoms or conditions induced by a pathogen. In some cases, symptoms or disease caused by a pathogen is prevented (or treated, e.g., reduced or ameliorated) by inhibiting replication of the pathogen following exposure of the subject to the pathogen. In the context of this disclosure, the term immunogenic composition will be understood to encompass compositions that are intended for administration to a subject or population of subjects for the purpose of eliciting a protective or palliative immune response against the virus (that is, vaccine compositions or vaccines).

The term “purification” (e.g., with respect to a pathogen or a composition containing a pathogen) refers to the process of removing components from a composition, the presence of which is not desired. Purification is a relative term, and does not require that all traces of the undesirable component be removed from the composition. In the context of vaccine production, purification includes such processes as centrifugation, dialization, ion-exchange chromatography, and size-exclusion chromatography, affinity-purification or precipitation. Thus, the term “purified” does not require absolute purity; rather, it is intended as a relative term. Thus, for example, a purified virus preparation is one in which the virus is more enriched than it is in its generative environment, for instance within a cell or population of cells in which it is replicated naturally or in an artificial environment. A preparation of substantially pure viruses can be purified such that the desired virus or viral component represents at least 50% of the total protein content of the preparation. In certain embodiments, a substantially pure virus will represent at least 60%, at least 70%, at least 80%, at least 85%, at least 90%, or at least 95% or more of the total protein content of the preparation.

An “isolated” biological component (such as a virus, nucleic acid molecule, protein or organelle) has been substantially separated or purified away from other biological components in the cell and/or organism in which the component occurs or is produced. Viruses and viral components, e.g., proteins, which have been “isolated” include viruses, and proteins, purified by standard purification methods. The term also embraces viruses and viral components (such as viral proteins) prepared by recombinant expression in a host cell.

An “antigen” is a compound, composition, or substance that can stimulate the production of antibodies and/or a T cell response in an animal, including compositions that are injected, absorbed or otherwise introduced into an animal. The term “antigen” includes all related antigenic epitopes. The term “epitope” or “antigenic determinant” refers to a site on an antigen to which B and/or T cells respond. The “dominant antigenic epitopes” or “dominant epitope” are those epitopes to which a functionally significant host immune response, e.g., an antibody response or a T-cell response, is made. Thus, with respect to a protective immune response against a pathogen, the dominant antigenic epitopes are those antigenic moieties that when recognized by the host immune system result in protection from disease caused by the pathogen. The term “T-cell epitope” refers to an epitope that when bound to an appropriate MHC molecule is specifically bound by a T cell (via a T cell receptor). A “B-cell epitope” is an epitope that is specifically bound by an antibody (or B cell receptor molecule). An antigen can also affect the innate immune response.

An “immune response” is a response of a cell of the immune system, such as a B cell, T cell, or monocyte, to a stimulus. An immune response can be a B cell response, which results in the production of specific antibodies, such as antigen specific neutralizing antibodies. An immune response can also be a T cell response, such as a CD4+ response or a CD8+ response. In some cases, the response is specific for a particular antigen (that is, an “antigen-specific response”). An immune response can also include the innate response. If the antigen is derived from a pathogen, the antigen-specific response is a “pathogen-specific response.” A “protective immune response” is an immune response that inhibits a detrimental function or activity of a pathogen, reduces infection by a pathogen, or decreases symptoms (including death) that result from infection by the pathogen. A protective immune response can be measured, for example, by the inhibition of viral replication or plaque formation in a plaque reduction assay or ELISA-neutralization assay, or by measuring resistance to pathogen challenge in vivo.

The immunogenic compositions disclosed herein are suitable for preventing, ameliorating and/or treating disease caused by infection of the virus.

By “reduce” or other forms of the word, such as “reducing” or “reduction,” is meant lowering of an event or characteristic (e.g., viral infection). It is understood that this is typically in relation to some standard or expected value, in other words it is relative, but that it is not always necessary for the standard or relative value to be referred to. For example, “reduces viral infection” means decreasing the amount of virus relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As used herein, “treatment” refers to obtaining beneficial or desired clinical results. Beneficial or desired clinical results include, but are not limited to, any one or more of: alleviation of one or more symptoms (such as infection), diminishment of extent of infection, stabilized (i.e., not worsening) state of infection, preventing or delaying spread of the infection, preventing or delaying occurrence or recurrence of infection, and delay or slowing of infection progression.

The term “patient” preferably refers to a human in need of treatment with an antibiotic or treatment for any purpose, and more preferably a human in need of such a treatment to treat viral infection. However, the term “patient” can also refer to non-human animals, preferably mammals such as dogs, cats, horses, cows, pigs, sheep and non-human primates, among others, that are in need of treatment with antibiotics.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

In addition, where features or aspects of the inventions are described in terms of Markush groups or other grouping of alternatives, those skilled in the art will recognize that the invention is also thereby described in terms of any individual member or subgroup of members of the Markush group or other group.

### General Description

RSV has four major structural proteins (glycoprotein [G], fusion [F] protein, Nucleoprotein [N] and M₂₋₁) which are responsible for induction of humoral and cell mediated immune responses in the infected individual. Humoral (or antibody mediated) immunity is required for neutralizing/limiting the virus spread, whereas, cell mediated immunity is required for clearance of the virus from the body of the infected individual. G and F are surface proteins and induce both neutralizing antibodies and T cell mediated immune responses. N and M₂₋₁ are internal proteins and contribute in induction of T cell response.

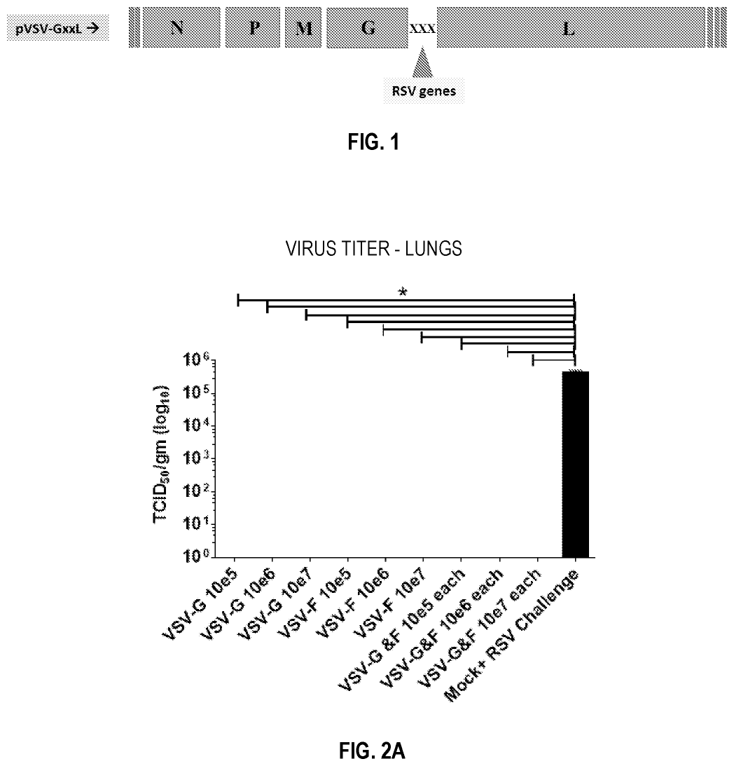

Four types of recombinant VSVs have been developed, each individually expressing one of the four above mentioned antigenic structural proteins (modified or unmodified) between glycoprotein (G) and polymerase (L) protein genes of the rVSV vector (FIG. 1). For expression of G protein, in addition to the cloning of wildtype G protein gene in the rVSV, a codon optimized version of the gene has been cloned. Codon optimization of a gene enables higher expression of the vaccine antigen (G protein, in this case). Therefore, from the same dose of the vaccine, a codon optimized gene expressing VSV produces significantly higher levels of the antigen protein resulting in dose amplification, so that the required dose of the rVSV can be significantly reduced. Further, in the context of RSV infection, G protein is produced in two forms (membrane bound [mG] and secretory [sG] forms). rVSVs expressing both forms have been produced. Further, RSV-G protein has been cloned with (Table 1) and pre-clinical in vivo efficacy studies have been conducted in the cotton rat animal model.

It is noted that viruses other than RSV can be used with the rVSV platforms disclosed herein. Examples of other viruses are known to those of skill in the art and include other respiratory (human and animal) viruses such as, human metapneumo virus, influenza, and bRSV.

TABLE 1

| S. | Name of the | Characteristic of the RSV | Rationale for expression in the |
|---|---|---|---|
| No. | recombinant | G protein | rVSV vector |
| 1 | rVSV-G | Wildtype RSV-G protein | G protein is the receptor binding |
| | | | ligand of the RSV and immunogenic |
| | | | because of presence of antigenic |
| | | | epitopes |
| 2 | rVSV-cG | Codon-optimized RSV- G | Codon optimization enhances |
| | | protein (full length with 298 | expression of the G protein resulting |
| | | amino acid [AA] length). | in dose sparing/amplification effect. |
| 3 | rVSV-mG | Codon-optimized RSV- G | Membrane bound G protein is more |
| | | protein stabilized to express | immunogenic than secretory G |
| | | only membrane bound form | protein. |
| | | by mutating second start | |
| | | codon at amino acid (AA) | |
| | | position 48, from Methionine | |
| | | to Isoleucine (M48I). | |
| 4 | rVSV-G | Codon-optimized RSV- G | Expected to be attenuated because of |
| | (C186S) | protein with disrupted | the disrupted receptor binding |
| | | receptor binding domain, | domain and possibly increasing the |
| | | CX3C motif, in the ‘cysteine | immunogenicity of the G protein. |
| | | noose’ of the central | |
| | | conserved domain of the G | |
| | | protein. | |
| 5 | rVSV- SecG | Codon-optimized 250 AA | To elucidate the purported role of |
| | | sized mRSV- G (starting | Sec-G as ‘decoy’ antigen |
| | | from second start codon at | |
| | | AA position 48/with | |
| | | truncated cytoplasmic tail | |
| | | and part of the | |
| | | transmembrane domain) | |
| 6 | rVSV-GΔNg | Codon optimized RSV- G | Few reports have shown that |
| | | protein with deletion of five | unglycosylated/prokaryotically |
| | | putative N-glycosylation | expressed G protein was more |
| | | sites by mutation of | immunogenic than glycosylated form. |
| | | Aspargine residue to alanine. | |
| 7 | rVSV- | Codon optimized ‘membrane | We predicted that membrane-bound |
| | mGΔNg | bound’ RSV- G protein (as | partially unglycosylated G is more |
| | | in rVSV-mG) with deletion | immunogenic than rVSV-GΔNg |
| | | of five putative N- | |
| | | glycosylation sites by | |
| | | mutation of Aspargine | |
| | | residue to alanine. | |
| 8 | rVSV-G | 28 AA long peptide | Shown to be immunogenic in other |
| | (aa163-190) | comprising of ‘central | expression systems as it compasses |
| | | conserved domain’ of the G | the most conserved region of the G |
| | | protein | protein including receptor binding |
| | | | CX3C motif |
| 9 | rVSV-G | 101 AA long peptide | Shown to be immunogenic in other |
| | (aa130-230) | comprising of ‘central | expression systems as it compasses |
| | | conserved domain’ of the G | the most conserved region of the G |
| | | protein | protein including receptor binding |
| | | | CX3C motif |

RSV F protein is involved in the fusion of the virus to the cell membrane of the infected cell and has a higher number of neutralizing epitopes, antigenic sites and T-cell epitopes than G protein, thus, making it an attractive vaccine candidate. F protein exists in two different structural conformations, pre-fusion and post-fusion (Pre-F and Post-F), and Pre-F has been shown to be more immunogenic than Post-F. Therefore, wildtype F and Pre-F genes have been cloned in rVSV (Table 2). The codon-optimized F gene in rVSV can also be cloned. Disclosed herein are various formats of F-protein, including codon-optimized F protein, pre-fusion conformation stabilized F-protein, and post-fusion F protein. The F protein can be wildtype or codon-optimized.

TABLE 2

| S. | Name of the | Characteristic of the RSV | Rationale for expression in the |
|---|---|---|---|
| No. | recombinant | F protein | rVSV vector |
| 1 | rVSV-F | Wildtype RSV-F protein | F protein is responsible for the fusion |
| | | | of the virus with host cell and has |
| | | | more number of neutralizing and |
| | | | CTL epitopes. |
| 2 | rVSV-Pre-F- | Codon-optimized RSV- F | Codon optimization enhances |
| | Foldon | protein with mutations in the | expression of the F protein resulting |
| | | F gene leading to stabilizing | in dose sparing/amplification effect. |
| | | the protein in Pre-F | Further, stabilization of the |
| | | conformation. | conformation in pre-fusion state |
| | | | enables it to induce highly protective |
| | | | immune response. |
| 3 | rVSV-Pre-F | Codon-optimized full-length | Codon optimization enhances |
| | | RSV- F protein with | expression of the F protein resulting |
| | | mutations in the F gene | in dose sparing/amplification effect. |
| | | leading to stabilizing the | Further, stabilization of the |
| | | protein in Pre-F | conformation in pre-fusion state |
| | | conformation. | enables it to induce highly protective |
| | | | immune response. |
| 4 | rVSV-Post F | Codon-optimized RSV- F | Post-fusion F protein is shown to |
| | | protein ectodomain | induce protective immunity in few |
| | | conformation. | studies. |
| 5 | rVSV-HEK- | Codon-optimized full-length | Codon optimization enhances |
| | Pre-F | RSV- F protein with | expression of the F protein resulting |
| | | mutations in the F gene | in dose sparing/amplification effect. |
| | | leading to stabilizing the | Further, stabilization of the |
| | | protein in Pre-F | conformation in pre-fusion state |
| | | conformation with HEK | enables it to induce highly protective |
| | | assignments. | immune response. |

Further, N and M₂₋₁ proteins have been shown to contain several putative sites of T-cell epitopes inducing cell mediated immunity, which is responsible for clearance of the infective RSV virus from the body. Therefore, rVSVs expressing M₂₋₁ and different segments of the N gene have been cloned and recovered (Table 3).

TABLE 3

| S. | Name of the | Characteristic of the RSV | Rationale for expression in the |
|---|---|---|---|
| No. | recombinant | N or M2-1 protein | rVSV vector |
| 1 | rVSV-NΔ3 | 238 AA length amino terminal | Comprises of two putative T-cell |
| | | domain (NTD) of the N protein | epitopes |
| 2 | rVSV-NΔ3-l | 254 AA length NTD and 16 AA | Comprises of five putative T-cell |
| | | of the carboxylic terminal | epitopes |
| | | domain (CTD) downstream of | |
| | | the NTD and CTD junction | |
| | | of the N protein | |
| 3 | rVSV-N- | 71 AA region of CTD | Comprises of two putative T-cell |
| | CTL-2 | | epitopes |
| 4 | rVSV-N- | 38 AA region of NTD and | Comprises of four putative T-cell |
| | CTL-4 | CTD junction | epitopes |
| 5 | rVSV-M₂₋₁ | Full-length wild type RSV- | Shown to possess CTL epitopes |
| | | M₂₋₁ protein | |

When a human or non-human animal is challenged by a foreign organism/pathogen the challenged individual responds by launching an immune response which may be protective. This immune response is characterized by the coordinated interaction of the innate and acquired immune response systems.

The innate immune response forms the first line of defense against a foreign organism/pathogen. An innate immune response can be triggered within minutes of infection in an antigen-independent, but pathogen-dependent, manner. The innate, and indeed the adaptive, immune system can be triggered by the recognition of pathogen associated molecular patterns unique to microorganisms by pattern recognition receptors present on most host cells. Once triggered the innate system generates an inflammatory response that activates the cellular and humoral adaptive immune response systems.

The adaptive immune response becomes effective over days or weeks and provides the antigen specific responses needed to control and usually eliminate the foreign organism/pathogen. The adaptive response is mediated by T cells (cell mediated immunity) and B cells (antibody mediated or humoral immunity) that have developed specificity for the pathogen. Once activated these cells have a long lasting memory for the same pathogen.

The ability of an individual to generate immunity to foreign organisms/pathogens, thereby preventing or at least reducing the chance of infection by the foreign organism/pathogen, is a powerful tool in disease control and is the principle behind vaccination.

Vaccines function by preparing the immune system to mount a response to a pathogen. Typically, a vaccine comprises an antigen, which is a foreign organism/pathogen or a toxin produced by an organism/pathogen, or a portion thereof, that is introduced into the body of a subject to be vaccinated in a non-toxic, and/or non-pathogenic form. The antigen in the vaccine causes the subject's immune system to be “primed” or “sensitized” to the organism/pathogen from which the antigen is derived. Subsequent exposure of the immune system of the subject to the organism/pathogen or toxin results in a rapid and robust specific immune response, that controls or destroys the organism/pathogen or toxin before it can multiply and infect or damage enough cells in the host organism to cause disease symptoms.

### Compositions

Disclosed herein are multiple rVSVs expressing one of the four different antigenic proteins (in natural or modified conformation) of RSV, which have been shown to be efficacious in a cotton rat animal model, with or without combining with an adjuvant expressing rVSV (rVSV-Hsp70). It has been demonstrated that when delivered intranasally, rVSVs expressing RSV proteins induce protective immunity in vaccinated cotton rats against wildtype RSV challenge.

Specifically, disclosed herein are compositions comprising a recombinant viral vector and one or more respiratory syncytial virus (RSV) proteins. The recombinant viral vector can be selected from recombinant viral vectors known to those of skill in the art. Non-limiting examples of vectors that can be used include viral-based vectors, such as those described in Lundstrom et al. (Vaccines 2016, 4, 39), hereby incorporated by reference in its entirety for its teaching concerning viral vectors (e.g., retrovirus, adenovirus, adeno-associated virus, lentivirus, HMPV, PIV). Examples of rVSV that can be used include, but are not limited to the expression of G and F in one vector, G and N sequences or an expression of an RSV gene and HSP as adjuvant. HSP can be human or other.

As mentioned above and in Example 1, there are four categories of RSV proteins which can be used in the compositions disclosed herein. It is noted that RSV can be from any source, such as human, bovine, etc. The RSV proteins include the G protein, the F protein, the M2-1 protein, and the N protein. Further, the G protein is present in two forms, the membrane bound (mG) and secretory (sG) forms. Either form can be used with the compositions and methods disclosed herein. These proteins can be used alone in the composition, or can be presented together to increase the antigenic response. For example, the G protein can be coupled with N, M2-1, or F proteins. The mG protein can be coupled with N, M2-1, or F proteins. Any of these proteins can be combined in any possible permutation for use in an immunogenic composition or vaccine. The RSV proteins used in the compositions and vaccines disclosed herein can be full length, or can be functional immunogenic fragments that retain their immunogenicity when administered to a subject. One of skill in the art will readily understand how to obtain immunogenic fragments of an RSV protein.

Furthermore, the proteins disclosed herein can be codon optimized. For example, the codon optimization of G and pre-fusion conformation stabilized F leads to higher and more stable expression of these proteins. Sequences are listed in the sequences listing. “Codon optimization” is defined as modifying a nucleic acid sequence for enhanced expression in the cells of the vertebrate of interest, e.g. human, by replacing at least one, more than one, or a significant number, of codons of the native sequence with codons that are more frequently or most frequently used in the genes of that vertebrate. Various species exhibit particular bias for certain codons of a particular amino acid.

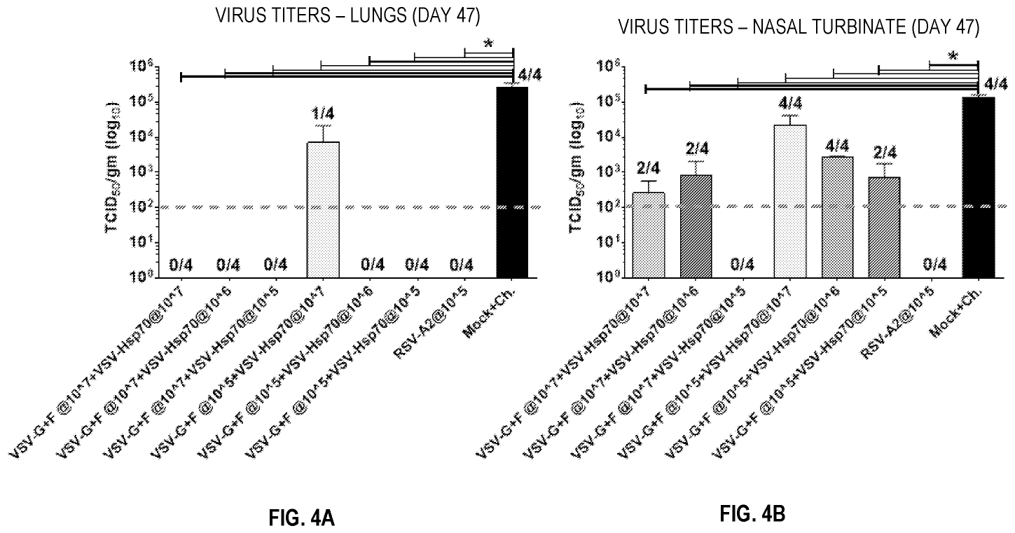

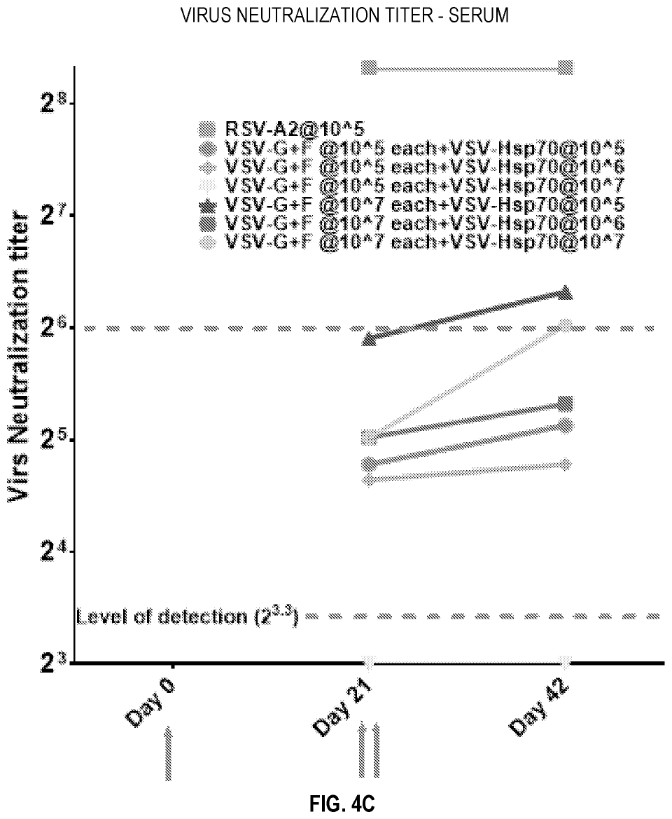

The composition disclosed herein can also comprise one or more adjuvants. As used herein, “adjuvant” is understood as an aid or contributor to increase the efficacy or potency of a vaccine or in the prevention, amelioration, or cure of disease by increasing the efficacy or potency of a therapeutic agent as compared to a vaccine or agent administered without the adjuvant. An increase in the efficacy or potency can include a decrease in the amount of vaccine or agent to be administered, a decrease in the frequency and/or number of doses to be administered, or a more rapid or robust response to the agent or vaccine (i.e., higher antibody titer). The adjuvant can be HSP70 (see FIG. 4), but may also include alumn, detoxified monophosphoryl lipid A (MPLA), detoxified saponin derivative QS-21 or other pattern recognition receptor agonists including NLP and TLR agonists. Other variants of HSP70 will have a similar effect, whether they are from a different species or mutated as long as the binding domain is intact.

Described herein are vaccines comprising a composition of this invention in a carrier wherein the vaccine is protective against RSV infection. The term “immunogenic carrier” as used herein can refer to a first polypeptide or fragment, variant, or derivative thereof which enhances the immunogenicity of a second polypeptide or fragment, variant, or derivative thereof. An “immunogenic carrier” can be fused, to or conjugated/coupled to the desired polypeptide or fragment thereof. See, e.g., European Patent No. EP 0385610 B1, which is incorporated herein by reference in its entirety for its teaching of fusing, conjugating or coupling a polypeptide to a carrier. An example of an “immunogenic carrier” is PLGA.

The vaccine composition of the present invention may also be co-administered with antigens from other pathogens as a multivalent vaccine.

### Methods of Use and Administration

Also disclosed herein are methods of using the immunogenic compositions and vaccines disclosed herein. For example, disclosed are methods of eliciting an immune response against RSV in a subject, the method comprising administering to the subject a composition or vaccine as disclosed herein. The immune response can be protective against RSV, for example.

Also disclosed is a method of reducing symptoms or duration of RSV in a subject, the method comprising the steps of: (a) providing a composition of any of claims 1 to 15 or the vaccine of claim 16; and (b) administering said composition or vaccine to the subject, thereby reducing symptoms or duration of RSV.

Further disclosed is a method of stimulating an immune response in a subject, the method comprising: administering to said subject a composition or vaccine as disclosed herein.

The vaccines disclosed herein can be administered in a variety of ways, and at a variety of doses. For example, intranasal route, orally, intramuscular route, intradermal and subcutaneous injection as well as application by ocular, vaginal and anal route.

In one example, a single dose of the immunogenic composition or vaccine can be given, wherein the composition comprises about 1×10⁵ or more particles (which also are referred to as particle units (pu)) of the composition, e.g., about 1×10⁶ or more particles, about 1×10′ or more particles, about 1×10⁸ or more particles, about 1×10⁹ or more particles, or about 3×10⁸ or more particles of the composition. Alternatively, or in addition, a single dose of the composition comprises about 3×10¹⁴ particles or less of the immunogenic composition, e.g., about 1×10¹³ particles or less, about 1×10¹² particles or less, about 3×10¹¹ particles or less, about 1×10¹¹ particles or less, about 1×10¹⁰ particles or less, or about 1×10⁹ particles or less of the immunogenic composition. Thus, a single dose of immunogenic composition can comprise a quantity of particles of the immunogenic composition in a range defined by any two of the aforementioned values. For example, a single dose of immunogenic composition can comprise 1×10⁵-1×10¹⁴ particles, 1×10⁷-1×10¹² particles, 1×10⁸-1×10¹¹ particles, 3×10⁸-3×10″ particles, 1×10⁹-1×10¹² particles, 1×10⁹-1×10¹¹ particles, 1×10⁹-1×10¹⁰ particles, or 1×10¹⁰-1×10¹² particles, of the immunogenic composition. In other words, a single dose of immunogenic composition can comprise, for example, about 1×10⁶ pu, 2×10⁶ pu, 4×10⁶ pu, 1×10⁷ pu, 2×10⁷ pu, 4×10⁷ pu, 1×10⁸ pu, 2×10⁸ pu, 3×10⁸ pu, 4×10⁸ pu, 1×10⁹ pu, 2×10⁹ pu, 3×10⁹ pu, 4×10⁹ pu, 1×10¹⁰ pu, 2×10¹⁰ pu, 3×10¹⁰ pu, 4×10¹⁰ pu, 1×10¹¹ pu, 2×10¹¹ pu, 3×10¹¹ pu, 4×10¹¹ pu, 1×10¹² pu, 2×10¹² pu, 3×10¹² pu, or 4×10¹² pu of the adenoviral vector.

The vaccine can be given in single doses, or two doses which are separated. For example, when two doses are given, they can be given 1, 2, 3, 4, 5, 6, 7, 8, 9, 10, 11, 12, 13, 14 or more days apart. The vaccine can be administered in a variety of ways known to those of skill in the art, such as intranasally.

### EXAMPLES

### Example 1: 10⁷ Pfu/Dose/Animal of the rVSVs Expressing Wild Type G and F Proteins Induced Protective Immunity in Cotton Rats

Since 10⁵ TCID₅₀/dose of the RSV induced protective immunity in cotton rats (n=4 per group), therefore, for relative comparison of rVSVs with the RSV immune efficacy of the rVSV-G and rVSV-F recombinants, 10⁵ pfu (plaque forming unit)/dose as the starting dose and also immunized with higher/10 fold incremental doses (10⁶ pfu/animal or 10⁷ pfu/animal) were evaluated. Cotton rats were immunized with either individual rVSV-G or rVSV-F recombinant or in combination (rVSV-G+F). The hypothesis was that rVSV induced protective effect is dose dependent and further, enhanced effect is possible by combining both G and F expressing rVSVs. Immunized animals were challenged with wild type RSV strain A2 (dose: 10⁵ TCID₅₀) four weeks after vaccination and euthanized the animals four days after challenge. Clearance of the challenge virus was evaluated (by titrating the amount of virus using a cell culture cytopathic effect based assay) from the lower and upper respiratory tract (LRT and URT) represented by homogenates of the lungs and nasal passage respectively (collected on the day of euthanization) and virus neutralizing (VN) antibody levels (by cell culture based virus neutralization test) in the serum sample collected on the day of challenge. These studies demonstrated that non-invasive mucosal delivery of the rVSV-G or F by intranasal route was more effective than parental (by subcutaneous) route of administration. Therefore, for all subsequent studies, intranasal immunization method was employed. Further, it was also shown that 10⁵ pfu/animal of either rVSV-G or rVSV-F was effective in clearance of challenge virus from the LRT but not URT along with lower VN antibody levels. Therefore, the objective of this study was to extend the protection to URT and enhance the VN antibody levels by employing higher dose and combined vaccination strategy.

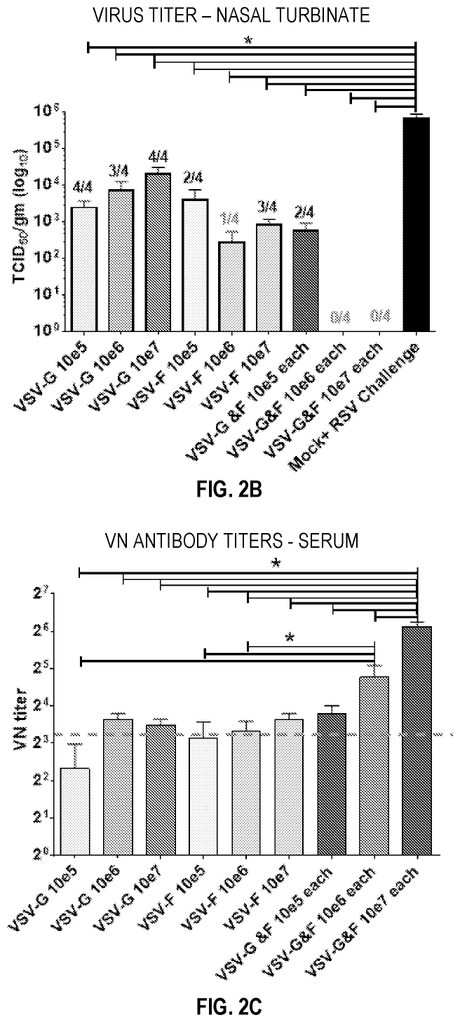

The results indicated that, higher (each rVSV at 10⁷ pfu/dose/cotton rat[CR]) and combined (rVSV-G+F) immunization strategy was effective in inducing protective immunity which could clear the challenge RSV from both LRT and URT (FIGS. 2a&b) along with higher levels of VN antibody levels (FIG. 2c).

These results and the comparison of VSV expressing either G or F with immunization results through immunization with purified G and post-fusion F protein (Table 4) demonstrate that VSV vectors deliver a better immune response.

### Example 2: Prime-Boost Immunization Regimen of rVSVs Expressing Wild Type G and F Proteins Induced Protective Immunity in Cotton Rats Along with Enhanced VN Titers

Though 10⁷ pfu dose of rVSV-G and rVSV-F combination was adequate to protect the immunized cotton rats from the challenge virus, virus neutralization (VN) antibody titers were still lower than RSV-A2 immunized animals (which showed higher VN titer titers, ≥2⁸). Therefore, to enhance the VN titers in rVSV immunized groups, it was hypothesized that by following prime-boost regimen of immunization strategy, VN titers can be significantly enhanced with high (10⁷ pfu) and possibly with low dose (10⁵ pfu) immunization as well. Therefore, cotton rats were immunized with either high dose or low dose of rVSVs, individually or in combination, and the booster dose was administered three weeks after primary immunization and the immunized cotton rats were challenged three weeks after booster immunization.

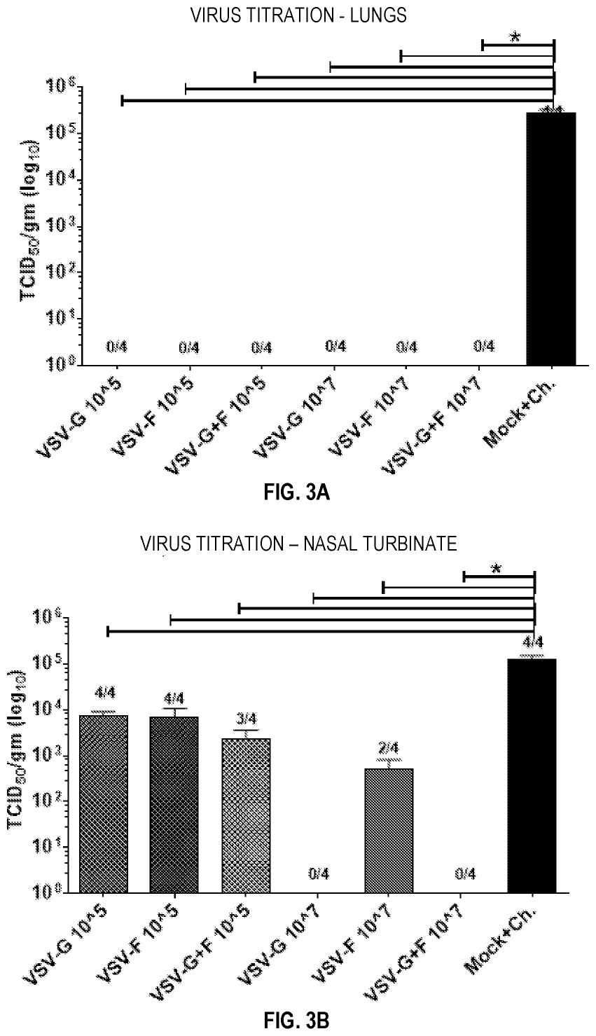

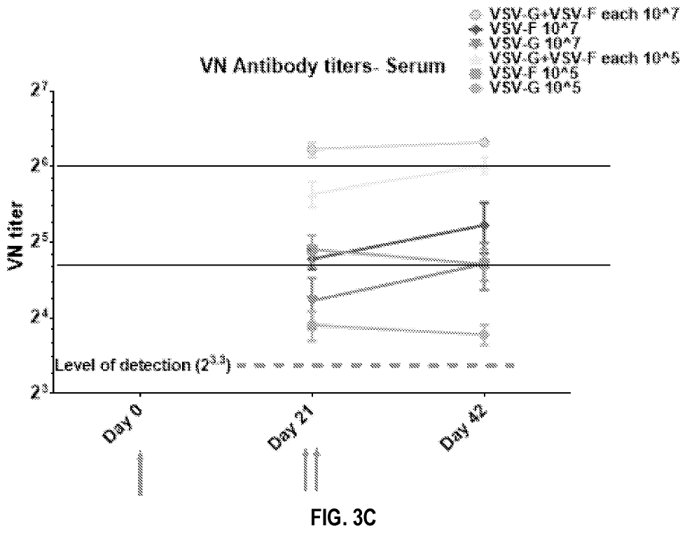

The results indicated that, at low dose immunization, neither individual nor combined rVSVs induced protective immunity in URT, and VN titers were also not considerably improved upon booster immunization. Whereas, in higher dose immunization groups, in all three groups VN antibodies were enhanced after booster immunization (FIG. 3c) along with complete protection of URT in rVSV-G and rVSV-G+F immunized groups (FIGS. 3a&b). Prime-boost regimen was effective in enhancing the VN titers by up to 40% after booster immunization. Thus, it was evident from this study that, prime-boost immunization enhanced protective immunity in the immunized animals (and possibly indicating an extended longevity [i. e., memory immune response] of the protection).

Immunization can also be improved through the use of a VSV expressing HSP70 which functions as an adjuvant (FIG. 4).

### Example 3: Coupling of an Adjuvant Expressing rVSV Along with Prime-Boost Immunization Regimen of rVSVs Expressing Wild Type G and F Proteins Induced Enhanced Protective Immunity in Cotton Rats

Though prime-boost immunization with rVSV-G+F enhanced the VN titers (titer: ˜2⁶), however, the VN titers in RSV-A2 immunized animals were significantly higher (titer: >2⁸). Therefore, with an objective to further enhancing the protective immunity in the rVSV-G+F immunized animals and to explore a possibility of extending the longevity of the protection, the vaccine rVSV candidates were combined with Hsp70 expressing rVSV (rVSV-Hsp-70). It has been demonstrated that rVSV-Hsp70 enhanced adjuvanticity of the vaccine antigen co-expressing rVSV (Ma, et al., 2014) resulting in enhanced mucosal immunity. Further the safe dose of rVSV-Hsp70 (i. e., ≤10⁷ pfu/dose/CR) has been shown in cotton rats. Therefore, in the present study, with an objective to identify the appropriate dose of rVSV-Hsp70 along with rVSV-G+F, cotton rats were immunized (following prime-boost regimen) with either high dose or low dose combination of rVSV-G+F and combined with one of the three doses (10⁵, 10⁶, or 10⁷ pfu/dose/CR) of the rVSV-Hsp70.

The results indicated that, 10⁵ pfu dose of the rVSV-Hsp70 was an appropriate dose along with high dose of the rVSV-G+F as there was complete protection of both LRT and URT (FIGS. 4a&b) along with enhanced VN titers by 33% (FIG. 4c).

It is clearly evident from the above studies that, prime-boost immunization of the 10⁷ pfu dose of each of rVSV-G and rVSV-F combination induced enhanced protective immunity in the cotton rat model. Further, efficacy of the combination (and possibly longevity of the protection) can be further enhanced by inclusion of the adjuvant expressing rVSV-Hsp70.

### Example 4: Codon-Optimized or Membrane-Bound Codon Optimized RSV G Protein Expressing rVSVs (rVSV-cG or rVSV-mG) were More Effective than Wild-Type G (rVSV-G) in Inducing Protective Immunity in the URT Along with Enhanced VN Titers

In order to identify an effective G protein candidate, several modifications were made to the G protein to enhance its immunogenicity as explained in table 1 (S. No. 2-9) and expressed the indicated G variant in the VSV vector and tested the efficacy in the cotton rats. Cotton rats were immunized with each of the seven recovered rVSV G variants, following the previously established strategy for the rVSV-G+F immunization studies (i. e., high dose [10⁷ pfu/dose/CR] and prime-boost immunization).

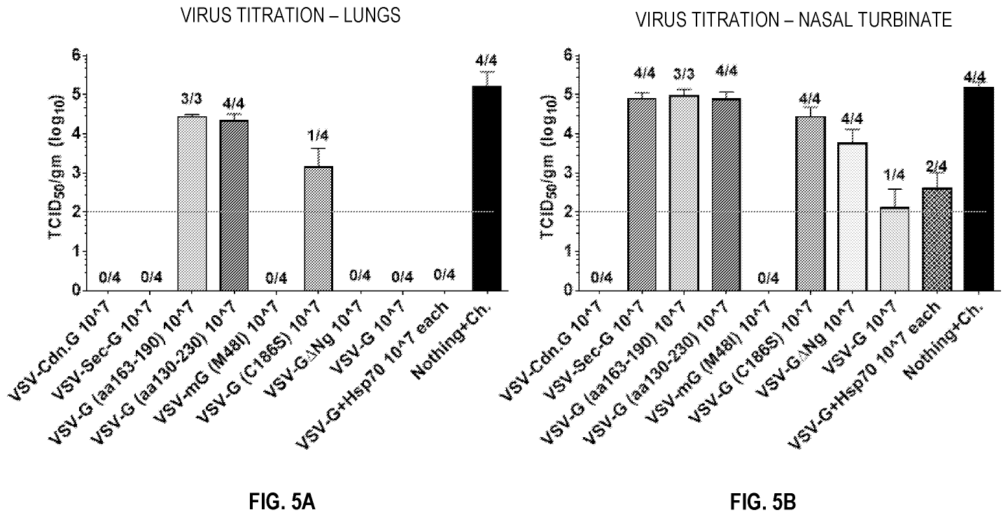

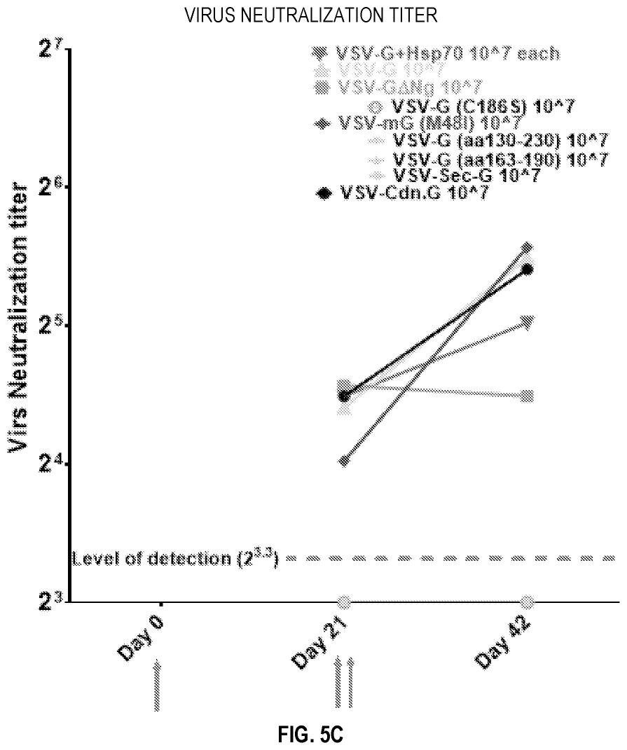

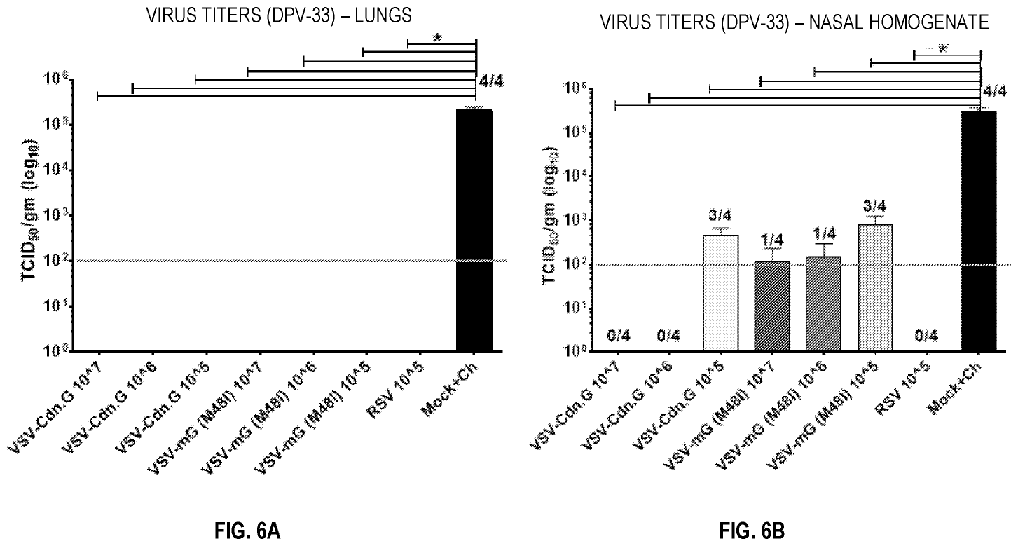

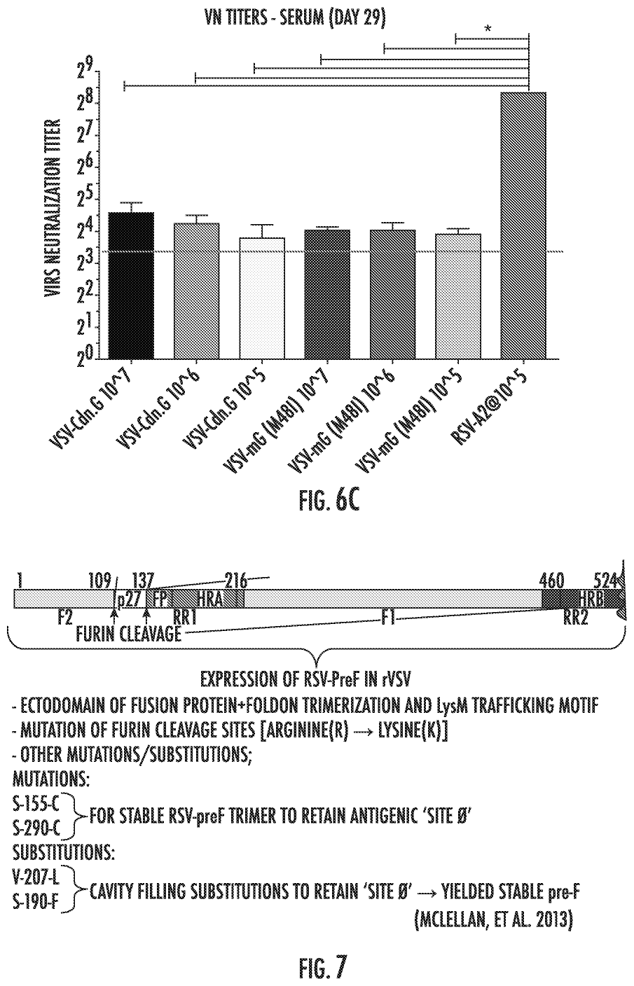

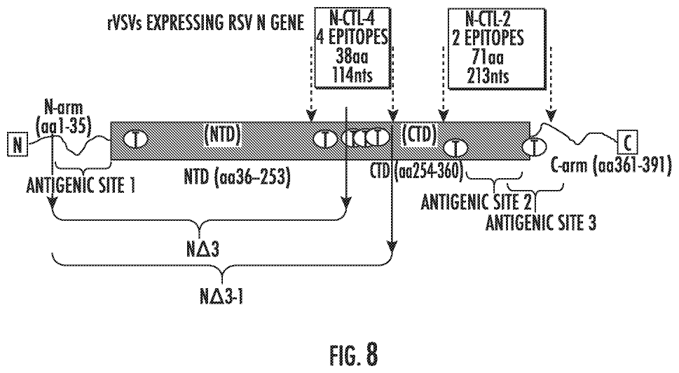

The results clearly indicated that among all the tested G variants, two recombinants (rVSV-cG and rVSV-mG) were successful in inducing protective immunity in the in the URT (FIGS. 5a&b) as well as enhanced VN titers compared to rVSV-G (FIG. 5c). These results demonstrated that, either expressing the codon-optimized G protein (which produces higher levels of both membrane bound as well as secretory forms) at higher level endogenously or expressing it exclusively in the membrane bound form (by excluding the ‘decoy’ effect of the secretory G), it is possible to induce protective immunity by RSV G protein alone. Further, single dose immunization with 10⁵, 10⁶, or 10⁷ dose of rVSV-cG or rVSV-mG was tested for effectiveness in eliciting protective immunity. The results demonstrated that higher dose (10⁷ pfu) of the either recombinant was effective in either completely protecting the URT (rVSV-cG) or reducing the challenge virus titer (rVSV-mG) (FIG. 6b). However, the VN titers in all the groups were low and comparable (FIG. 6c). These results demonstrated that, even though modified G recombinants (cG and mG) alone were effective in protecting both LRT and URT, however, to induce enhanced VN antibodies, prime-boost immunization regimen is essential.

TABLE 4
Immunization with G and F protein. G and post-fusion F protein
were expressed eucaryotically in 293F cells. Cotton rats were immunized
with 5 ug of purified protein in 200 ul alumn subcutaneously. Four weeks
later, blood was drawn to determine neutralizing antibody titers and
animals were challenged with 10⁵ TCID₅₀ RSV.
Four days later, virus titers were determined from lung and nasal tissue.
Post-fusion F is currently tested in clinical trials.

| | | | Neutralizing |
|---|---|---|---|
| | Nose | Lung | antibody |
| Naïve animals | 3.8 ± 0.2 | 3.4 ± 0.3 | 2⁰ |
| Immunized with G | 3.9 ± 0.4 | 3.9 ± 0.4 | 2⁰ |
| protein | | | |
| Immunized with | 0 | 0 | 2^{4.5} |
| post-F protein | | | |

SEQUENCES
Sequences of RSV genes expressed in the VSV expression system
SEQ ID NO: 1: RSV-G (Size: 897 nts)
ATGTCCAAAAACAAGGACCAACGCACCGCTAAGACATTAGAAAGGACCTGG
GACACTCTCAATCATTTATTATTCATATCATCGTGCTTATATAAGTTAAATCTTAAAT
CTGTAGCACAAATCACATTATCCATTCTGGCAATGATAATCTCAACTTCACTTATAA
TTGCAGCCATCATATTCATAGCCTCGGCAAACCACAAAGTCACACCAACAACTGCA
ATCATACAAGATGCAACAAGCCAGATCAAGAACACAACCCCAACATACCTCACCCA
GAATCCTCAGCTTGGAATCAGTCCCTCTAATCCGTCTGAAATTACATCACAAATCAC
CACCATACTAGCTTCAACAACACCAGGAGTCAAGTCAACCCTGCAATCCACAACAG
TCAAGACCAAAAACACAACAACAACTCAAACACAACCCAGCAAGCCCACCACAAA
ACAACGCCAAAACAAACCACCAAGCAAACCCAATAATGATTTTCACTTTGAAGTGT
TCAACTTTGTACCCTGCAGCATATGCAGCAACAATCCAACCTGCTGGGCTATCTGCA
AAAGAATACCAAACAAAAAACCAGGAAAGAAAACCACTACCAAGCCCACAAAAAA
ACCAACCCTCAAGACAACCAAAAAAGATCCCAAACCTCAAACCACTAAATCAAAGG
AAGTACCCACCACCAAGCCCACAGAAGAGCCAACCATCAACACCACCAAAACAAAC
ATCATAACTACACTACTCACCTCCAACACCACAGGAAATCCAGAACTCACAAGTCA
AATGGAAACCTTCCACTCAACTTCCTCCGAAGGCAATCCAAGCCCTTCTCAAGTCTC
TACAACATCCGAGTACCCATCACAACCTTCATCTCCACCCAACACACCACGCCAGTA
G
SEQ ID NO: 2: RSV-cG Icodon optimized G] (size :897 nts)
ATGAGCAAGAACAAGGACCAGCGGACCGCCAAGACCCTGGAGCGGACCTGG
GACACCCTGAACCACCTGCTGTTCATCAGCAGCTGCCTGTACAAGCTGAACCTGAAG
AGCGTGGCCCAGATCACCCTGAGCATCCTGGCCATGATCATCAGCACCAGCCTGATC
ATCGCCGCCATCATCTTCATCGCCAGCGCCAACCACAAGGTGACCCCCACCACCGCC
ATCATCCAGGACGCCACCAGCCAGATCAAGAACACCACCCCCACCTACCTGACCCA
GAACCCCCAGCTGGGCATCAGCCCCAGCAACCCCAGCGAGATCACCAGCCAGATCA
CCACCATCCTGGCCAGCACCACCCCCGGCGTGAAGAGCACCCTGCAGAGCACCACC
GTGAAGACCAAGAACACCACCACCACCCAGACCCAGCCCAGCAAGCCCACCACCAA
GCAGCGGCAGAACAAGCCTCCCAGCAAGCCCAACAACGACTTCCACTTCGAGGTGT
TCAACTTCGTGCCCTGCAGCATCTGCAGCAACAACCCCACCTGCTGGGCCATCTGCA
AGCGGATTCCCAACAAGAAGCCCGGCAAGAAGACCACCACCAAGCCCACCAAGAA
GCCCACCCTGAAGACCACCAAGAAGGACCCCAAGCCCCAGACCACCAAGAGCAAG
GAGGTGCCCACCACCAAGCCCACCGAGGAGCCCACCATCAACACCACCAAGACCAA
CATCATCACCACCCTGCTGACCAGCAACACCACCGGCAACCCCGAGCTGACCAGCC
AGATGGAGACCTTCCACAGCACCAGCAGCGAGGGCAACCCCAGCCCCAGCCAGGTG
AGCACCACCAGCGAGTACCCCAGCCAGCCCAGCAGCCCTCCCAACACCCCTCGGCA
GTAG
SEQ ID NO: 3: RSV-cmG [codon optimized membrane bound G]
(size: 897 nts)
ATGAGCAAGAACAAGGACCAGCGGACCGCCAAGACCCTGGAGCGGACCTGG
GACACCCTGAACCACCTGCTGTTCATCAGCAGCTGCCTGTACAAGCTGAACCTGAAG
AGCGTGGCCCAGATCACCCTGAGCATCCTGGCCATTATCATCAGCACCAGCCTGATC
ATCGCCGCCATCATCTTCATCGCCAGCGCCAACCACAAGGTGACCCCCACCACCGCC
ATCATCCAGGACGCCACCAGCCAGATCAAGAACACCACCCCCACCTACCTGACCCA
GAACCCCCAGCTGGGCATCAGCCCCAGCAACCCCAGCGAGATCACCAGCCAGATCA
CCACCATCCTGGCCAGCACCACCCCCGGCGTGAAGAGCACCCTGCAGAGCACCACC
GTGAAGACCAAGAACACCACCACCACCCAGACCCAGCCCAGCAAGCCCACCACCAA
GCAGCGGCAGAACAAGCCTCCCAGCAAGCCCAACAACGACTTCCACTTCGAGGTGT
TCAACTTCGTGCCCTGCAGCATCTGCAGCAACAACCCCACCTGCTGGGCCATCTGCA
AGCGGATTCCCAACAAGAAGCCCGGCAAGAAGACCACCACCAAGCCCACCAAGAA
GCCCACCCTGAAGACCACCAAGAAGGACCCCAAGCCCCAGACCACCAAGAGCAAG
GAGGTGCCCACCACCAAGCCCACCGAGGAGCCCACCATCAACACCACCAAGACCAA
CATCATCACCACCCTGCTGACCAGCAACACCACCGGCAACCCCGAGCTGACCAGCC
AGATGGAGACCTTCCACAGCACCAGCAGCGAGGGCAACCCCAGCCCCAGCCAGGTG
AGCACCACCAGCGAGTACCCCAGCCAGCCCAGCAGCCCTCCCAACACCCCTCGGCA
GTAG
SEQ ID NO: 4: RSV-G(C1865) (Size: 897 nts)
ATGAGCAAGAACAAGGACCAGCGGACCGCCAAGACCCTGGAGCGGACCTGG
GACACCCTGAACCACCTGCTGTTCATCAGCAGCTGCCTGTACAAGCTGAACCTGAAG
AGCGTGGCCCAGATCACCCTGAGCATCCTGGCCATGATCATCAGCACCAGCCTGATC
ATCGCCGCCATCATCTTCATCGCCAGCGCCAACCACAAGGTGACCCCCACCACCGCC
ATCATCCAGGACGCCACCAGCCAGATCAAGAACACCACCCCCACCTACCTGACCCA
GAACCCCCAGCTGGGCATCAGCCCCAGCAACCCCAGCGAGATCACCAGCCAGATCA
CCACCATCCTGGCCAGCACCACCCCCGGCGTGAAGAGCACCCTGCAGAGCACCACC
GTGAAGACCAAGAACACCACCACCACCCAGACCCAGCCCAGCAAGCCCACCACCAA
GCAGCGGCAGAACAAGCCTCCCAGCAAGCCCAACAACGACTTCCACTTCGAGGTGT
TCAACTTCGTGCCCTGCAGCATCTGCAGCAACAACCCCACCTGCTGGGCCATCTCCA
AGCGGATTCCCAACAAGAAGCCCGGCAAGAAGACCACCACCAAGCCCACCAAGAA
GCCCACCCTGAAGACCACCAAGAAGGACCCCAAGCCCCAGACCACCAAGAGCAAG
GAGGTGCCCACCACCAAGCCCACCGAGGAGCCCACCATCAACACCACCAAGACCAA
CATCATCACCACCCTGCTGACCAGCAACACCACCGGCAACCCCGAGCTGACCAGCC
AGATGGAGACCTTCCACAGCACCAGCAGCGAGGGCAACCCCAGCCCCAGCCAGGTG
AGCACCACCAGCGAGTACCCCAGCCAGCCCAGCAGCCCTCCCAACACCCCTCGGCA
GTAG
SEQ ID NO: 5: RSV-Sec G (756 nts)
ATGATCATCAGCACCAGCCTGATCATCGCCGCCATCATCTTCATCGCCAGCGC
CAACCACAAGGTGACCCCCACCACCGCCATCATCCAGGACGCCACCAGCCAGATCA
AGAACACCACCCCCACCTACCTGACCCAGAACCCCCAGCTGGGCATCAGCCCCAGC
AACCCCAGCGAGATCACCAGCCAGATCACCACCATCCTGGCCAGCACCACCCCCGG
CGTGAAGAGCACCCTGCAGAGCACCACCGTGAAGACCAAGAACACCACCACCACCC
AGACCCAGCCCAGCAAGCCCACCACCAAGCAGCGGCAGAACAAGCCTCCCAGCAA
GCCCAACAACGACTTCCACTTCGAGGTGTTCAACTTCGTGCCCTGCAGCATCTGCAG
CAACAACCCCACCTGCTGGGCCATCTGCAAGCGGATTCCCAACAAGAAGCCCGGCA
AGAAGACCACCACCAAGCCCACCAAGAAGCCCACCCTGAAGACCACCAAGAAGGA
CCCCAAGCCCCAGACCACCAAGAGCAAGGAGGTGCCCACCACCAAGCCCACCGAGG
AGCCCACCATCAACACCACCAAGACCAACATCATCACCACCCTGCTGACCAGCAAC
ACCACCGGCAACCCCGAGCTGACCAGCCAGATGGAGACCTTCCACAGCACCAGCAG
CGAGGGCAACCCCAGCCCCAGCCAGGTGAGCACCACCAGCGAGTACCCCAGCCAGC
CCAGCAGCCCTCCCAACACCCCTCGGCAGTAG
SEQ ID NO: 6: RSV-GΔNg (897nts)
ATGTCTAAAAACAAGGATCAGCGAACCGCCAAAACCCTGGAGCGTACATGG
GATACACTCAACCACCTTCTGTTCATATCTAGCTGCCTTTACAAACTTAATCTCAAAA
GCGTCGCCCAGATTACCCTCTCAATACTGGCTATGATAATCTCCACCTCTTTGATAAT
AGCCGCTATCATTTTCATAGCTTCTGCAAACCACAAAGTAACTCCAACCACAGCTAT
AATACAAGATGCCACCTCTCAGATTAAAAATACCACACCCACATATCTTACTCAGAA
TCCTCAATTGGGAATCAGCCCATCTAAgCCATCCGAGATTACTTCACAGATCACCAC
AATACTCGCATCCACAACACCAGGGGTCAAATCCACCCTGCAATCAACTACCGTGA
AAACTAAAAAgACCACTACAACACAGACTCAACCCAGCAAGCCTACAACAAAGCAA
CGCCAGAATAAGCCACCTTCTAAGCCAAACAATGATTTCCATTTTGAGGTCTTTAAT
TTCGTGCCTTGCTCTATATGTTCCAACAAgCCAACTTGCTGGGCCATTTGCAAACGCA
TCCCAAATAAGAAACCCGGTAAGAAAACCACAACCAAGCCAACTAAAAAGCCAACT
TTGAAGACTACCAAAAAGGACCCTAAGCCCCAGACAACTAAATCAAAAGAAGTCCC
AACTACTAAGCCAACTGAGGAACCAACAATAAAgACTACAAAAACCAACATCATCA
CAACCCTTCTTACTAGCAAgACTACTGGTAACCCCGAGCTGACAAGCCAGATGGAGA
CATTCCACAGTACAAGCAGCGAAGGAAACCCAAGCCCTAGTCAAGTGTCCACTACC
TCAGAATACCCCAGCCAGCCTTCCTCACCTCCTAACACACCCCGGCAATAG
SEQ ID NO: 7: RSV-mGΔNg (897nts)
cagcaatctcgagATGTCTAAAAACAAGGATCAGCGAACCGCCAAAACCCTGGAGC
GTACATGGGATACACTCAACCACCTTCTGTTCATATCTAGCTGCCTTTACAAACTTA
ATCTCAAAAGCGTCGCCCAGATTACCCTCTCAATACTGGCTATTATAATCTCCACCTC
TTTGATAATAGCCGCTATCATTTTCATAGCTTCTGCAAACCACAAAGTAACTCCAAC
CACAGCTATAATACAAGATGCCACCTCTCAGATTAAAAATACCACACCCACATATCT
TACTCAGAATCCTCAATTGGGAATCAGCCCATCTAAgCCATCCGAGATTACTTCACA
GATCACCACAATACTCGCATCCACAACACCAGGGGTCAAATCCACCCTGCAATCAA
CTACCGTGAAAACTAAAAAgACCACTACAACACAGACTCAACCCAGCAAGCCTACA
ACAAAGCAACGCCAGAATAAGCCACCTTCTAAGCCAAACAATGATTTCCATTTTGA
GGTCTTTAATTTCGTGCCTTGCTCTATATGTTCCAACAAgCCAACTTGCTGGGCCATT
TGCAAACGCATCCCAAATAAGAAACCCGGTAAGAAAACCACAACCAAGCCAACTAA
AAAGCCAACTTTGAAGACTACCAAAAAGGACCCTAAGCCCCAGACAACTAAATCAA
AAGAAGTCCCAACTACTAAGCCAACTGAGGAACCAACAATAAAgACTACAAAAACC
AACATCATCACAACCCTTCTTACTAGCAAgACTACTGGTAACCCCGAGCTGACAAGC
CAGATGGAGACATTCCACAGTACAAGCAGCGAAGGAAACCCAAGCCCTAGTCAAGT
GTCCACTACCTCAGAATACCCCAGCCAGCCTTCCTCACCTCCTAACACACCCCGGCA
ATAGcccgggttcat
SEQ ID NO: 8: RSV-G (aa 163-190) (84 nts)
TTCCACTTCGAGGTGTTCAACTTCGTGCCCTGCAGCATCTGCAGCAACAACCC
CACCTGCTGGGCCATCTGCAAGCGGATTCCC
SEQ ID NO: 9: RSV-G (aa 130-230) (303 nts)
ACCGTGAAGACCAAGAACACCACCACCACCCAGACCCAGCCCAGCAAGCCC
ACCACCAAGCAGCGGCAGAACAAGCCTCCCAGCAAGCCCAACAACGACTTCCACTT
CGAGGTGTTCAACTTCGTGCCCTGCAGCATCTGCAGCAACAACCCCACCTGCTGGGC
CATCTGCAAGCGGATTCCCAACAAGAAGCCCGGCAAGAAGACCACCACCAAGCCCA
CCAAGAAGCCCACCCTGAAGACCACCAAGAAGGACCCCAAGCCCCAGACCACCAA
GAGCAAGGAGGTGCCCACCACCAAGCCC
SEQ ID NO: 10: RSV-F (size: 1725 nts)
ATGGAGTTGCTAATCCTCAAAGCAAATGCAATTACCACAATCCTCACTGCAG
TCACATTTTGTTTTGCTTCTGGTCAAAACATCACTGAAGAATTTTATCAATCAACATG
CAGTGCAGTTAGCAAAGGCTATCTTAGTGCTCTGAGAACTGGTTGGTATACCAGTGT
TATAACTATAGAATTAAGTAATATCAAGAAAAATAAGTGTAATGGAACAGATGCTA
AGGTAAAATTGATAAAACAAGAATTAGATAAATATAAAAATGCTGTAACAGAATTG
CAGTTGCTCATGCAAAGCACACAAGCAACAAACAATCGAGCCAGAAGAGAACTACC
AAGGTTTATGAATTATACACTCAACAATGCCAAAAAAACCAATGTAACATTAAGCA
AGAAAAGGAAAAGAAGATTTCTTGGTTTTTTGTTAGGTGTTGGATCTGCAATCGCCA
GTGGCGTTGCTGTATCTAAGGTCCTGCACCTAGAAGGGGAAGTGAACAAGATCAAA
AGTGCTCTACTATCCACAAACAAGGCTGTAGTCAGCTTATCAAATGGAGTTAGTGTT
TTAACCAGCAAAGTGTTAGACCTCAAAAACTATATAGATAAACAATTGTTACCTATT
GTGAACAAGCAAAGCTGCAGCATATCAAATATAGAAACTGTGATAGAGTTCCAACA
AAAGAACAACAGACTACTAGAGATTACCAGGGAATTTAGTGTTAATGCAGGCGTAA
CTACACCTGTAAGCACTTACATGTTAACTAATAGTGAATTATTGTCATTAATCAATG
ATATGCCTATAACAAATGATCAGAAAAAGTTAATGTCCAACAATGTTCAAATAGTTA
GACAGCAAAGTTACTCTATCATGTCCATAATAAAAGAGGAAGTCTTAGCATATGTA
GTACAATTACCACTATATGGTGTTATAGATACACCCTGTTGGAAACTACACACATCC
CCTCTATGTACAACCAACACAAAAGAAGGGTCCAACATCTGTTTAACAAGAACTGA
CAGAGGATGGTACTGTGACAATGCAGGATCAGTATCTTTCTTCCCACAAGCTGAAAC
ATGTAAAGTTCAATCAAATCGAGTATTTTGTGACACAATGAACAGTTTAACATTACC
AAGTGAAGTAAATCTCTGCAATGTTGACATATTCAACCCCAAATATGATTGTAAAAT
TATGACTTCAAAAACAGATGTAAGCAGCTCCGTTATCACATCTCTAGGAGCCATTGT
GTCATGCTATGGCAAAACTAAATGTACAGCATCCAATAAAAATCGTGGAATCATAA
AGACATTTTCTAACGGGTGCGATTATGTATCAAATAAAGGGGTGGACACTGTGTCTG
TAGGTAACACATTATATTATGTAAATAAGCAAGAAGGTAAAAGTCTCTATGTAAAA
GGTGAACCAATAATAAATTTCTATGACCCATTAGTATTCCCCTCTGATGAATTTGAT
GCATCAATATCTCAAGTCAACGAGAAGATTAACCAGAGCCTAGCATTTATTCGTAAA
TCCGATGAATTATTACATAATGTAAATGCTGGTAAATCCACCACAAATATCATGATA
ACTACTATAATTATAGTGATTATAGTAATATTGTTATCATTAATTGCTGTTGGACTGC
TCTTATACTGTAAGGCCAGAAGCACACCAGTCACACTAAGCAAAGATCAACTGAGT
GGTATAAATAATATTGCATTTAGTAACTAA
SEQ ID NO: 11: RSV-Pre-F-Foldon (1941 nts)
ATGGAGCTGCTCATCCTGAAGGCCAACGCCATCACCACCATCCTCACCGCCG
TGACCTTCTGCTTCGCCAGCGGCCAGAATATCACAGAGGAATTTTATCAGTCTACTT
GTAGTGCCGTCAGTAAAGGATATCTGAGCGCTCTCAGAACAGGATGGTACACTAGT
GTTATTACAATAGAATTGTCAAATATCAAGAAAAATAAGTGCAACGGTACTGACGC
TAAGGTTAAGCTCATCAAACAGGAACTTGATAAATATAAGAACGCAGTTACAGAAC
TTCAGCTTCTTATGCAGTCCACACAAGCCACCAATAATAAAGCTAAAAAAGAACTCC
CACGGTTCATGAACTACACACTGAACAATGCAAAAAAAACCAACGTAACCCTTAGC
AAGAAAAAGAAAAAAAAGTTCCTTGGCTTCCTCCTCGGAGTAGGCAGCGCTATTGC
AAGTGGGGTAGCCGTGTGTAAGGTTTTGCATCTCGAAGGAGAAGTGAATAAAATAA
AGAGCGCCTTGCTGTCCACTAATAAGGCCGTAGTCAGCCTTAGCAATGGCGTATCCG
TTCTGACCTTTAAAGTACTGGATTTGAAGAACTACATCGATAAACAGCTTCTCCCCA
TTTTGAATAAGCAATCATGTTCTATCAGTAACATAGAAACCGTCATCGAATTCCAAC
AAAAAAACAATCGGCTTTTGGAAATAACTCGTGAATTTTCTGTAAACGCAGGCGTG
ACAACTCCCGTATCAACCTACATGTTGACCAATAGCGAACTGCTGTCACTCATTAAC
GACATGCCAATCACTAACGACCAGAAAAAACTTATGAGCAATAATGTACAGATTGT
AAGACAGCAAAGTTACAGCATAATGTGCATTATTAAGGAAGAAGTTTTGGCATACG
TTGTCCAACTCCCCCTTTATGGGGTCATTGATACCCCCTGCTGGAAGCTGCATACTA
GCCCATTGTGTACTACCAACACCAAAGAGGGTAGTAACATATGCCTCACCAGAACT
GACCGAGGCTGGTACTGTGATAATGCTGGAAGTGTCAGTTTCTTTCCTCAAGCAGAG
ACCTGCAAAGTTCAGTCCAACCGCGTGTTCTGTGATACAATGAACTCCCTGACACTC
CCTAGCGAAGTCAACCTTTGTAACGTCGATATATTTAATCCTAAATACGATTGTAAG
ATCATGACTTCAAAAACTGACGTATCCTCTTCCGTTATTACTTCTTTGGGTGCCATAG
TTAGTTGCTACGGCAAAACAAAATGCACCGCATCTAATAAAAACAGAGGAATTATT
AAGACATTTTCAAATGGTTGCGACTACGTTAGTAATAAAGGTGTAGATACAGTAAGT
GTTGGTAACACCCTCTATTACGTGAACAAACAGGAAGGGAAAAGCCTGTACGTGAA
AGGGGAGCCCATAATCAACTTCTACGACCCCCTTGTATTTCCTAGTGATGAATTTGA
CGCCTCCATCAGTCAAGTGAACGAAAAGATCAACCAAAGCCTTGCTTTCATCCGCAA
ATCCGATGAGTTGCTCCACAATATTAAAGGCTCGGGATATATACCGGAGGCCCCGC
GAGATGGTCAAGCTTATGTGCGCAAAGACGGTGAGTGGGTCTTGTTATCTACATTTT
TGGGTAACACTAATAGTGGAGGTAGCACGACGACAATTACTAATAATAACTCGGGA
ACTAACTCAAGCTCCACTACCTACACTGTCAAATCTGGTGATACATTGTGGGGCATA
AGTCAAAGATATGGTATTTCAGTAGCCCAAATTCAATCGGCGAATAATTTAAAGAG
CACAATAATTTACATAGGCCAGAAGCTCGTCCTGACAGGTTCCGCCTCGTCAACCAA
TAGCGGAGGCAGCAACAACAGTGCTTCAACGACACCCACCACCTCGGTTACTCCTG
CTAAGCCAACAAGTCAAACAACT
SEQ ID NO: 12: hCdn. RSV-Pre-F (1725 nts)
ATGGAACTTCTTATATTGAAGGCAAACGCAATCACCACCATTTTGACTGCGGT
TACATTCTGTTTCGCCTCAGGTCAAAATATTACAGAAGAATTCTACCAGAGCACATG
CTCAGCGGTATCAAAGGGTTACTTGTCAGCCCTTAGGACCGGATGGTATACCTCTGT
AATAACAATAGAACTTTCAAACATTAAAAAAAATAAGTGCAACGGGACCGATGCAA
AAGTTAAACTGATCAAGCAAGAACTGGACAAGTATAAAAACGCAGTCACTGAACTT
CAACTTCTTATGCAGTCCACGCAAGCCACTAATAATAAGGCTAAGAAAGAACTGCC
AAGGTTTATGAACTATACCCTGAACAACGCGAAGAAGACTAATGTCACGTTGTCAA
AAAAGAAAAAGAAAAAATTCCTGGGGTTCCTGCTCGGAGTAGGCAGTGCAATCGCG
TCTGGAGTAGCCGTATGTAAAGTATTGCACCTTGAAGGAGAAGTAAACAAAATAAA
GAGCGCTCTGCTCTCTACGAACAAAGCTGTTGTAAGTCTGAGCAATGGCGTCTCAGT
CCTGACATTTAAAGTTCTTGATTTGAAAAATTATATTGACAAACAACTCCTCCCTATC
CTCAACAAACAGTCTTGCTCTATTTCAAATATTGAGACAGTTATCGAATTTCAGCAA
AAAAACAATAGGCTCCTTGAAATCACACGAGAATTTTCTGTAAACGCTGGAGTCAC
AACACCAGTATCTACGTATATGCTCACCAATTCCGAACTTCTTTCATTGATAAATGA
TATGCCCATAACAAACGACCAGAAAAAATTGATGTCCAATAATGTCCAAATCGTTC
GCCAACAGAGCTATTCTATCATGTGTATAATAAAAGAGGAAGTTCTCGCTTACGTTG
TCCAACTGCCGCTGTACGGGGTGATTGACACACCTTGCTGGAAACTTCATACTAGCC
CTCTGTGCACGACTAACACCAAGGAAGGATCAAATATCTGCCTCACGCGAACTGAC
AGGGGTTGGTACTGTGATAACGCTGGTTCCGTGTCATTTTTTCCTCAAGCTGAGACG
TGTAAAGTACAGTCCAATCGAGTTTTCTGCGATACTATGAACTCACTCACCTTGCCG
TCAGAGGTGAACCTCTGTAACGTAGATATATTTAACCCGAAATACGACTGTAAGATT
ATGACTTCAAAGACCGATGTGTCAAGCTCCGTCATTACCTCCTTGGGAGCAATTGTT
TCTTGCTATGGTAAGACGAAGTGCACTGCGAGCAACAAGAATCGCGGTATCATCAA
GACGTTCTCCAACGGATGCGATTATGTAAGTAACAAGGGAGTTGACACGGTGAGTG
TAGGGAACACGTTGTACTATGTAAACAAGCAGGAGGGGAAGTCCTTGTATGTCAAG
GGCGAACCTATTATCAACTTCTACGACCCATTGGTGTTCCCTAGTGACGAGTTTGAT
GCTAGTATTTCCCAGGTCAACGAGAAGATAAACCAAAGTTTGGCTTTCATTAGGAAG
AGCGATGAGCTTCTCCACAATGTGAACGCCGGGAAGAGTACGACTAATATTATGAT
CACAACCATCATAATCGTCATTATCGTTATTTTGCTCTCACTGATTGCAGTCGGACTT
CTGCTGTACTGCAAAGCTCGCAGTACCCCAGTCACGCTTTCCAAGGACCAACTTTCA
GGCATTAATAACATCGCATTTTCTAATTAA
SEQ ID NO: 13: hCdn. RSV-Post-F (1509 nts)
ATGGAACTTTTGATACTGAAGGCGAACGCCATAACGACGATCCTGACAGCTG
TAACTTTTTGCTTCGCGAGCGGTCAAAACATAACCGAGGAATTTTATCAGTCAACGT
GCTCTGCTGTTAGCAAAGGATATCTCAGCGCACTCAGGACGGGCTGGTACACGTCA
GTCATAACGATTGAGCTGTCTAATATCAAGAAGAACAAATGCAACGGAACGGACGC
CAAAGTCAAGCTCATAAAACAAGAATTGGACAAGTACAAGAATGCTGTGACGGAGC
TTCAGCTCTTGATGCAGTCCACCCAAGCGACGAATAATAGAGCGAGGAGAGAGCTC
CCAAGATTTATGAACTATACACTGAACAATGCAAAGAAGACTAATGTGACCCTTAG
CAAGAAAAGAAAAAGAAGAGCGATTGCAAGTGGAGTGGCTGTGTCAAAGGTCCTG
CACCTTGAAGGTGAGGTGAACAAGATTAAATCCGCGCTGCTTTCTACGAACAAAGC
TGTCGTTAGTTTGTCCAATGGCGTTTCAGTGCTCACTTCCAAGGTATTGGATTTGAAG
AATTATATTGACAAACAGCTCCTTCCGATTGTTAATAAACAGAGTTGCTCAATTTCT
AACATCGAAACTGTCATAGAGTTTCAGCAGAAGAACAATCGGCTCTTGGAAATAAC
AAGGGAGTTTTCAGTCAACGCCGGGGTAACAACACCCGTGTCCACATACATGCTGA
CAAACTCCGAGTTGCTCTCTCTTATCAACGACATGCCAATTACAAACGACCAGAAGA
AATTGATGTCCAACAACGTCCAAATCGTACGACAGCAGTCTTATTCCATTATGAGTA
TTATTAAGGAAGAGGTATTGGCTTATGTAGTACAACTCCCCTTGTACGGGGTAATAG
ACACCCCCTGTTGGAAACTGCATACGAGTCCCCTGTGTACAACCAATACGAAGGAG
GGCTCCAATATATGTTTGACAAGAACTGACCGCGGCTGGTACTGTGATAATGCTGGT
AGTGTTAGCTTCTTTCCACAAGCGGAGACTTGCAAGGTACAATCTAATCGGGTTTTC
TGCGATACGATGAACTCTCTGACTCTGCCGAGTGAGGTCAACCTGTGCAACGTGGAC
ATATTCAATCCGAAGTACGATTGTAAAATTATGACATCCAAGACAGATGTAAGCAG
CTCTGTTATTACGTCACTGGGCGCTATTGTGTCATGCTACGGTAAGACTAAATGTAC
CGCATCCAATAAAAACAGGGGGATTATTAAAACCTTCAGCAACGGATGCGATTATG
TCAGCAATAAGGGCGTGGATACCGTATCCGTTGGCAATACTCTCTATTACGTAAATA
AACAGGAAGGCAAATCTCTCTATGTTAAAGGCGAACCTATAATCAATTTTTACGATC
CGCTTGTATTCCCTTCCGATGAATTCGATGCCTCTATCTCTCAAGTTAACGAAAAAAT
CAATCAATCTCTGGCATTTATTAGGAAGTCAGATGAACTCCTA
SEQ ID NO: 14: hCdn. RSV-HEK-Pre-F (1725 nts)
ATGGAATTGCTCATTTTGAAAGCTAATGCTATAACAACAATACTCACGGCTGT
AACTTTTTGCTTTGCCTCTGGTCAAAACATAACGGAAGAGTTTTATCAGTCAACGTG
TTCAGCCGTATCAAAAGGGTATCTTAGCGCACTGCGCACTGGATGGTACACGTCTGT
GATTACCATTGAACTCAGTAATATCAAGGAAAATAAATGCAACGGCACTGATGCAA
AAGTCAAGCTCATAAAACAGGAGCTTGACAAGTACAAAAATGCGGTTACAGAACTC
CAGCTCCTTATGCAATCTACCCCAGCAACCAACAACAAAGCCAAGAAGGAGCTGCC
CAGGTTTATGAACTATACACTTAACAACGCGAAGAAAACCAATGTCACTCTCAGTA
AAAAGAAAAAAAAGAAGTTCTTGGGGTTCCTTCTCGGTGTTGGAAGCGCCATTGCA
AGCGGTGTAGCAGTTTGCAAAGTTCTCCACCTTGAGGGGGAGGTGAACAAAATTAA
ATCTGCCCTCCTCTCAACTAACAAAGCCGTCGTCAGCTTGAGTAACGGCGTAAGCGT
ACTCACTTTCAAAGTTCTCGATCTGAAGAACTATATTGATAAACAGCTGCTCCCAAT
ACTGAACAAGCAGTCATGCAGCATCAGCAACATTGAAACCGTGATAGAGTTCCAGC
AGAAAAATAATAGGCTTTTGGAGATAACTCGGGAGTTTTCAGTCAACGCGGGTGTA
ACAACGCCAGTTTCCACGTATATGCTGACAAACAGTGAGCTCCTGAGCCTGATAAAT
GATATGCCAATCACAAACGATCAGAAAAAACTCATGTCCAATAACGTTCAGATAGT
ACGGCAACAGAGTTACAGCATAATGTGCATAATTAAAGAGGAGGTCCTGGCTTATG
TTGTCCAGCTTCCACTGTACGGGGTTATAGATACCCCATGTTGGAAGCTCCATACAT
CTCCCCTGTGTACTACTAACACCAAGGAGGGAAGCAATATATGTTTGACTCGCACTG
ACAGGGGTTGGTACTGTGATAATGCCGGGTCCGTGAGCTTTTTTCCGCAGGCTGAAA
CTTGCAAGGTGCAATCTAACCGAGTGTTCTGTGACACTATGAATTCTCTGACTCTCC
CGTCAGAAGTAAACTTGTGTAATGTCGACATATTTAACCCTAAATACGATTGTAAGA
TCATGACAAGCAAAACAGACGTCTCAAGTTCTGTCATAACAAGCTTGGGCGCGATT
GTGTCCTGTTATGGTAAAACCAAATGCACGGCGTCCAACAAAAATAGGGGCATTAT
TAAAACTTTTTCCAACGGCTGTGATTACGTCTCCAATAAAGGAGTGGATACGGTCTC
AGTTGGGAATACTCTGTACTATGTTAACAAACAAGAGGGCAAGTCTCTTTATGTGAA
AGGGGAACCGATTATAAACTTTTACGACCCGCTTGTGTTCCCGTCCGATGAGTTCGA
TGCGAGTATTTCCCAAGTCAACGAGAAGATAAACCAGTCCCTCGCGTTTATCCGCAA
AAGTGACGAGCTCCTTCATAACGTTAATGCTGGTAAGTCCACTACGAACATCATGAT
CACAACAATTATCATAGTCATTATTGTTATACTGCTTAGCCTGATCGCTGTAGGGTTG
CTCTTGTACTGTAAAGCGAGGTCTACCCCAGTTACCCTTAGTAAAGACCAATTGAGT
GGGATCAACAACATTGCGTTTTCCAATTGA
SEQ ID NO: 15: RSV-NΔ3 (714 nts)
CAACTTCTGTCATCCAGCAAATACACCATCCAACGGAGCACAGGAGATAGTA
TTGATACTCCTAATTATGATGTGCAGAAACACATCAATAAGTTATGTGGCATGTTAT
TAATCACAGAAGATGCTAATCATAAATTCACTGGGTTAATAGGTATGTTATATGCGA
TGTCTAGGTTAGGAAGAGAAGACACCATAAAAATACTCAGAGATGCGGGATATCAT
GTAAAAGCAAATGGAGTAGATGTAACAACACATCGTCAAGACATTAATGGAAAAGA
AATGAAATTTGAAGTGTTAACATTGGCAAGCTTAACAACTGAAATTCAAATCAACAT
TGAGATAGAATCTAGAAAATCCTACAAAAAAATGCTAAAAGAAATGGGAGAGGTA
GCTCCAGAATACAGGCATGACTCTCCTGATTGTGGGATGATAATATTATGTATAGCA
GCATTAGTAATAACTAAATTAGCAGCAGGGGACAGATCTGGTCTTACAGCCGTGATT
AGGAGAGCTAATAATGTCCTAAAAAATGAAATGAAACGTTACAAAGGCTTACTACC
CAAGGACATAGCCAACAGCTTCTATGAAGTGTTTGAAAAACATCCCCACTTTATAGA
TGTTTTTGTTCATTTTGGTATAGCACAATCTTCTACCAGAGGTGGCAGTAGAGTTGA
AGGGATTTTTGCAGGATTGTTTATGAATGCCTATGGTGCA
SEQ ID NO: 16: RSV-NΔ3-1 (762 nts)
CAACTTCTGTCATCCAGCAAATACACCATCCAACGGAGCACAGGAGATAGTA
TTGATACTCCTAATTATGATGTGCAGAAACACATCAATAAGTTATGTGGCATGTTAT
TAATCACAGAAGATGCTAATCATAAATTCACTGGGTTAATAGGTATGTTATATGCGA
TGTCTAGGTTAGGAAGAGAAGACACCATAAAAATACTCAGAGATGCGGGATATCAT
GTAAAAGCAAATGGAGTAGATGTAACAACACATCGTCAAGACATTAATGGAAAAGA
AATGAAATTTGAAGTGTTAACATTGGCAAGCTTAACAACTGAAATTCAAATCAACAT
TGAGATAGAATCTAGAAAATCCTACAAAAAAATGCTAAAAGAAATGGGAGAGGTA
GCTCCAGAATACAGGCATGACTCTCCTGATTGTGGGATGATAATATTATGTATAGCA
GCATTAGTAATAACTAAATTAGCAGCAGGGGACAGATCTGGTCTTACAGCCGTGATT
AGGAGAGCTAATAATGTCCTAAAAAATGAAATGAAACGTTACAAAGGCTTACTACC
CAAGGACATAGCCAACAGCTTCTATGAAGTGTTTGAAAAACATCCCCACTTTATAGA
TGTTTTTGTTCATTTTGGTATAGCACAATCTTCTACCAGAGGTGGCAGTAGAGTTGA
AGGGATTTTTGCAGGATTGTTTATGAATGCCTATGGTGCAGGGCAAGTGATGTTACG
GTGGGGAGTCTTAGCAAAATCAGTTAAAAAT
SEQ ID NO: 17: RSV-CTL-2 (213 nts)
GCAGGATTCTACCATATATTGAACAACCCAAAAGCATCATTATTATCTTTGAC
TCAATTTCCTCACTTCTCCAGTGTAGTATTAGGCAATGCTGCTGGCCTAGGCATAAT
GGGAGAGTACAGAGGTACACCGAGGAATCAAGATCTATATGATGCAGCAAAGGCAT
ATGCTGAACAACTCAAAGAAAATGGTGTGATTAACTACAGTGTACTA
SEQ ID NO: 18: RSV-N-CTL-4 (114 nts)
TCTACCAGAGGTGGCAGTAGAGTTGAAGGGATTTTTGCAGGATTGTTTATGA
ATGCCTATGGTGCAGGGCAAGTGATGTTACGGTGGGGAGTCTTAGCAAAATCAGTT
AAAAAT
SEQ ID NO: 19: RSV-M2-1 (585 nts)
ATGTCACGAAGGAATCCTTGCAAATTTGAAATTCGAGGTCATTGCTTAAATG
GTAAGAGGTGTCATTTTAGTCATAATTATTTTGAATGGCCACCCCATGCACTGCTTGT
AAGACAAAACTTTATGTTAAACAGAATACTTAAGTCTATGGATAAAAGTATAGATA
CCTTATCAGAAATAAGTGGAGCTGCAGAGTTGGACAGAACAGAAGAGTATGCTCTT
GGTGTAGTTGGAGTGCTAGAGAGTTATATAGGATCAATAAACAATATAACTAAACA
ATCAGCATGTGTTGCCATGAGCAAACTCCTCACTGAACTCAATAGTGATGATATCAA
AAAGCTGAGGGACAATGAAGAGCTAAATTCACCCAAGATAAGAGTGTACAATACTG
TCATATCATATATTGAAAGCAACAGGAAAAACAATAAACAAACTATCCATCTGTTA
AAAAGATTGCCAGCAGACGTATTGAAGAAAACCATCAAAAACACATTGGATATCCA
TAAGAGCATAACCATCAACAACCCAAAAGAATCAACTGTTAGTGATACAAATGACC
ATGCCAAAAATAATGATACTACCTGA
SEQ ID NO: 20: Human HSP-70 (1926 nts or 642 aa)
ATGGCCAAAGCCGCGGCAGTCGGCATCGACCTGGGCACCACCTACTCCTGCG
TGGGGGTGTTCCAACACGGCAAGGTGGAGATCATCGCCAACGACCAGGGCAACCGC
ACCACCCCCAGCTACGTGGCCTTCACGGACACCGAGCGGCTCATCGGGGATGCGGC
CAAGAACCAGGTGGCGCTGAACCCGCAGAACACCGTGTTTGACGCGAAGCGCCTGA
TTGGCCGCAAGTTCGGCGACCCGGTGGTGCAGTCGGACATGAAGCACTGGCCTTTCC
AGGTGATCAACGACGGAGACAAGCCCAAGGTGCAGGTGAGCTACAAGGGGGAGAC
CAAGGCATTCTACCCCGAGGAGATCTCGTCCATGGTGCTGACCAAGATGAAGGAGA
TCGCCGAGGCGTACCTGGGCTACCCGGTGACCAACGCGGTGATCACCGTGCCGGCC
TACTTCAACGACTCGCAGCGCCAGGCCACCAAGGATGCGGGTGTGATCGCGGGGCT
CAACGTGCTGCGGATCATCAACGAGCCCACGGCCGCCGCCATCGCCTACGGCCTGG
ACAGAACGGGCAAGGGGGAGCGCAACGTGCTCATCTTTGACCTGGGCGGGGGCACC
TTCGACGTGTCCATCCTGACGATCGACGACGGCATCTTCGAGGTGAAGGCCACGGCC
GGGGACACCCACCTGGGTGGGGAGGACTTTGACAACAGGCTGGTGAACCACTTCGT
GGAGGAGTTCAAGAGAAAACACAAGAAGGACATCAGCCAGAACAAGCGAGCCGTG
AGGCGGCTGCGCACCGCCTGCGAGAGGGCCAAGAGGACCCTGTCGTCCAGCACCCA
GGCCAGCCTGGAGATCGACTCCCTGTTTGAGGGCATCGACTTCTACACGTCCATCAC
CAGGGCGAGGTTCGAGGAGCTGTGCTCCGACCTGTTCCGAAGCACCCTGGAGCCCG
TGGAGAAGGCTCTGCGCGACGCCAAGCTGGACAAGGCCCAGATTCACGACCTGGTC
CTGGTCGGGGGCTCCACCCGCATCCCCAAGGTGCAGAAGCTGCTGCAGGACTTCTTC
AACGGGCGCGACCTGAACAAGAGCATCAACCCCGACGAGGCTGTGGCCTACGGGGC
GGCGGTGCAGGCGGCCATCCTGATGGGGGACAAGTCCGAGAACGTGCAGGACCTGC
TGCTGCTGGACGTGGCTCCCCTGTCGCTGGGGCTGGAGACGGCCGGAGGCGTGATG
ACTGCCCTGATCAAGCGCAACTCCACCATCCCCACCAAGCAGACGCAGATCTTCACC
ACCTACTCCGACAACCAACCCGGGGTGCTGATCCAGGTGTACGAGGGCGAGAGGGC
CATGACGAAAGACAACAATCTGTTGGGGCGCTTCGAGCTGAGCGGCATCCCTCCGG
CCCCCAGGGGCGTGCCCCAGATCGAGGTGACCTTCGACATCGATGCCAACGGCATC
CTGAACGTCACGGCCACGGACAAGAGCACCGGCAAGGCCAACAAGATCACCATCAC
CAACGACAAGGGCCGCCTGAGCAAGGAGGAGATCGAGCGCATGGTGCAGGAGGCG
GAGAAGTACAAAGCGGAGGACGAGGTGCAGCGCGAGAGGGTGTCAGCCAAGAACG
CCCTGGAGTCCTACGCCTTCAACATGAAGAGCGCCGTGGAGGATGAGGGGCTCAAG
GGCAAGATCAGCGAGGCGGACAAGAAGAAGGTGCTGGACAAGTGTCAAGAGGTCA
TCTCGTGGCTGGACGCCAACACCTTGGCCGAGAAGGACGAGTTTGAGCACAAGAGG
AAGGAGCTGGAGCAGGTGTGTAACCCCATCATCAGCGGACTGTACCAGGGTGCCGG
TGGTCCCGGGCCTGGGGGCTTCGGGGCTCAGGGTCCCAAGGGAGGGTCTGGGTCAG
GCC CCACCATTGAGGAGGTAGATTAG
Sequence to express RSV-G and F genes in tandem
SEQ ID NO: 21: hCdn. RSV G-2A-F (2682 nts) (G and F genes
separated by 2A peptide sequence)
ATGTCCAAAAACAAGGATCAACGAACGGCTAAAACACTGGAAAGAACTTGG
GATACTCTTAATCACCTTCTTTTCATCAGCTCCTGTTTGTATAAGTTGAACTTGAAAA
GTGTAGCACAAATTACCTTGTCAATTCTGGCTATGATTATTTCCACTAGTTTGATCAT
TGCTGCGATTATATTTATTGCTTCTGCAAATCATAAGGTAACCCCGACTACAGCGAT
CATTCAGGACGCTACAAGTCAAATAAAGAACACCACACCGACGTACTTGACCCAGA
ATCCCCAGCTTGGCATCAGTCCTTCTAACCCTTCTGAAATCACCTCCCAAATCACCA
CTATCCTTGCGTCTACCACACCTGGAGTAAAGAGTACATTGCAGTCTACTACCGTTA
AGACCAAGAACACAACCACAACTCAAACGCAGCCATCTAAGCCAACTACCAAACAG
CGGCAAAATAAACCTCCATCTAAACCGAATAACGATTTTCACTTTGAAGTATTCAAC
TTTGTTCCCTGCTCAATTTGCAGCAATAATCCGACCTGCTGGGCTATATGTAAGCGG
ATACCAAATAAAAAGCCAGGAAAGAAAACTACAACAAAACCTACGAAGAAGCCTA
CACTGAAGACCACAAAAAAAGACCCAAAACCCCAGACAACCAAGTCCAAGGAAGT
TCCCACTACTAAGCCCACTGAAGAGCCTACCATAAATACCACCAAGACAAACATCA
TAACCACCTTGCTCACCTCTAATACTACCGGAAACCCTGAGCTCACTTCCCAAATGG
AAACGTTCCATTCAACTAGTAGTGAGGGCAACCCGAGTCCCAGCCAGGTCTCTACA
ACCTCAGAATACCCCTCCCAACCTAGTTCACCCCCAAATACTCCACGGCAGGGATCC
GGAGAGGGAAGAGGAAGTTTGCTGACATGTGGAGATGTGGAGGAAAATCCCGGTCC
AATGGAGCTTCTGATCCTGAAAGCTAACGCTATTACTACTATACTTACCGCCGTAAC
ATTCTGCTTCGCCTCCGGACAAAACATCACAGAAGAGTTCTATCAATCCACGTGCAG
CGCTGTGTCTAAGGGCTATCTGAGCGCATTGAGAACGGGGTGGTATACTTCCGTAAT
TACTATAGAGCTGTCAAACATTAAGAAAAACAAGTGTAACGGTACCGACGCTAAAG
TAAAGCTCATCAAGCAGGAGCTGGATAAATACAAAAATGCTGTCACTGAACTCCAG
CTTCTTATGCAATCTACCCAAGCAACCAACAACCGGGCTAGGCGCGAATTGCCCAG
GTTCATGAATTATACATTGAACAACGCCAAAAAGACTAATGTAACCCTCAGCAAGA
AACGCAAGAGGCGGTTCCTGGGATTTCTTCTCGGAGTAGGTTCCGCTATAGCGTCCG
GAGTAGCGGTCTCAAAAGTATTGCATCTGGAAGGCGAAGTTAACAAAATTAAGAGC
GCGCTCCTCAGCACCAACAAGGCGGTAGTCAGCCTCAGCAACGGCGTATCTGTTCTC
ACATCTAAAGTTTTGGACCTGAAAAACTATATAGACAAGCAGTTGCTTCCGATAGTA
AATAAGCAATCATGTTCCATTTCAAACATAGAAACGGTTATCGAGTTTCAACAGAAA
AATAATAGATTGCTTGAGATCACAAGAGAGTTCTCTGTCAATGCAGGTGTGACTACG
CCGGTCAGCACATATATGCTCACGAATAGTGAACTGCTGAGTCTTATAAATGATATG
CCGATTACTAATGACCAAAAAAAGCTCATGAGCAACAATGTCCAAATCGTTCGACA
ACAAAGTTACTCTATCATGAGCATCATCAAAGAGGAGGTTCTCGCATATGTCGTGCA
GCTTCCGTTGTATGGTGTAATAGATACCCCGTGCTGGAAGCTGCACACCTCTCCACT
GTGCACAACCAATACTAAAGAGGGGTCTAATATCTGTCTCACGAGAACGGATCGAG
GATGGTACTGCGATAACGCCGGTAGTGTGAGCTTCTTCCCCCAGGCTGAAACCTGTA
AGGTACAGAGTAACAGGGTATTCTGTGACACTATGAACTCACTCACACTGCCAAGT
GAAGTGAACCTTTGTAACGTTGACATATTTAATCCCAAGTACGACTGCAAAATCATG
ACAAGCAAAACCGACGTTTCCTCAAGCGTCATAACGAGTTTGGGTGCTATAGTAAGT
TGCTATGGGAAAACCAAGTGCACGGCATCCAATAAGAACAGAGGGATCATAAAAAC
GTTCTCCAACGGATGTGACTATGTGTCAAACAAGGGGGTTGATACGGTATCAGTTGG
AAATACCCTTTATTATGTCAACAAGCAGGAAGGAAAGAGCCTCTATGTAAAAGGCG
AACCCATAATCAATTTTTATGACCCACTCGTATTCCCTAGTGATGAGTTCGATGCCTC
TATTAGCCAGGTAAATGAGAAGATCAACCAGAGTTTGGCCTTTATCCGCAAATCTGA
CGAGCTGCTCCATAATGTCAATGCAGGGAAAAGTACGACTAATATCATGATTACTAC
GATTATTATCGTCATCATCGTCATCCTCTTGAGTCTTATAGCGGTAGGGCTCCTGCTC
TACTGTAAAGCGCGCTCTACCCCTGTGACGCTGTCCAAAGATCAACTTTCTGGCATA
AACAACATTGCCTTTAGTAATTAA
SEQ ID NO: 22: VSV (Indiana strain)
ACGAAGACAAACAAACCATTATTATCATTAAAAGGCTCAGGAGAAACTTTAA
CAGTAATCAAAATGTCTGTTACAGTCAAGAGAATCATTGACAACACAGTCATAGTTC
CAAAACTTCCTGCAAATGAGGATCCAGTGGAATACCCGGCAGATTACTTCAGAAAA
TCAAAGGAGATTCCTCTTTACATCAATACTACAAAAAGTTTGTCAGATCTAAGAGGA
TATGTCTACCAAGGCCTCAAATCCGGAAATGTATCAATCATACATGTCAACAGCTAC
TTGTATGGAGCATTAAAGGACATCCGGGGTAAGTTGGATAAAGATTGGTCAAGTTTC
GGAATAAACATCGGGAAAGCAGGGGATACAATCGGAATATTTGACCTTGTATCCTT
GAAAGCCCTGGACGGCGTACTTCCAGATGGAGTATCGGATGCTTCCAGAACCAGCG
CAGATGACAAATGGTTGCCTTTGTATCTACTTGGCTTATACAGAGTGGGCAGAACAC
AAATGCCTGAATACAGAAAAAAGCTCATGGATGGGCTGACAAATCAATGCAAAATG
ATCAATGAACAGTTTGAACCTCTTGTGCCAGAAGGTCGTGACATTTTTGATGTGTGG
GGAAATGACAGTAATTACACAAAAATTGTCGCTGCAGTGGACATGTTCTTCCACATG
TTCAAAAAACATGAATGTGCCTCGTTCAGATACGGAACTATTGTTTCCAGATTCAAA
GATTGTGCTGCATTGGCAACATTTGGACACCTCTGCAAAATAACCGGAATGTCTACA
GAAGATGTAACGACCTGGATCTTGAACCGAGAAGTTGCAGATGAAATGGTCCAAAT
GATGCTTCCAGGCCAAGAAATTGACAAGGCCGATTCATACATGCCTTATTTGATCGA
CTTTGGATTGTCTTCTAAGTCTCCATATTCTTCCGTCAAAAACCCTGCCTTCCACTTC
TGGGGGCAATTGACAGCTCTTCTGCTCAGATCCACCAGAGCAAGGAATGCCCGACA
GCCTGATGACATTGAGTATACATCTCTTACTACAGCAGGTTTGTTGTACGCTTATGC
AGTAGGATCCTCTGCCGACTTGGCACAACAGTTTTGTGTTGGAGATAACAAATACAC
TCCAGATGATAGTACCGGAGGATTGACGACTAATGCACCGCCACAAGGCAGAGATG
TGGTCGAATGGCTCGGATGGTTTGAAGATCAAAACAGAAAACCGACTCCTGATATG
ATGCAGTATGCGAAAAGAGCAGTCATGTCACTGCAAGGCCTAAGAGAGAAGACAAT
TGGCAAGTATGCTAAGTCAGAATTTGACAAATGACCCTATAATTCTCAGATCACCTA
TTATATATTATGCTACATATGAAAAAAACTAACAGATATCATGGATAATCTCACAAA
AGTTCGTGAGTATCTCAAGTCCTACTCTCGTCTAGATCAGGCGGTAGGAGAGATAGA
TGAGATCGAAGCACAACGAGCTGAAAAGTCCAATTATGAGTTGTTCCAAGAGGACG
GAGTGGAAGAGCATACTAGGCCCTCTTATTTTCAGGCAGCAGATGATTCTGACACAG
AATCTGAACCAGAAATTGAAGACAATCAAGGCTTGTATGTACCAGATCCGGAAGCT
GAGCAAGTTGAAGGCTTTATACAGGGGCCTTTAGATGACTATGCAGATGAGGACGT
GGATGTTGTATTCACTTCGGACTGGAAACAGCCTGAGCTTGAATCCGACGAGCATGG
AAAGACCTTACGGTTGACATTGCCAGAGGGTTTAAGTGGAGAGCAGAAATCCCAGT
GGCTTTTGACGATTAAAGCAGTCGTTCAAAGTGCCAAACACTGGAATCTGGCAGAG
TGCACATTTGAAGCATCGGGAGAAGGGGTCATCATAAAAAAGCGCCAGATAACTCC
GGATGTATATAAGGTCACTCCAGTGATGAACACACATCCGTACCAATCAGAAGCCG
TATCAGATGTTTGGTCTCTCTCAAAGACATCCATGACTTTCCAACCCAAGAAAGCAA
GTCTTCAGCCTCTCACCATATCCTTGGATGAATTGTTCTCATCTAGAGGAGAATTCAT
CTCTGTCGGAGGTAACGGACGAATGTCTCATAAAGAGGCCATCCTGCTCGGTCTGAG
GTACAAAAAGTTGTACAATCAGGCGAGAGTCAAATATTCTCTGTAGACTATGAAAA
AAAGTAACAGATATCACAATCTAAGTGTTATCCCAATCCATTCATCATGAGTTCCTT
AAAGAAGATTCTCGGTCTGAAGGGGAAAGGTAAGAAATCTAAGAAATTAGGGATCG
CACCACCCCCTTATGAAGAGGACACTAGCATGGAGTATGCTCCGAGCGCTCCAATTG
ACAAATCCTATTTTGGAGTTGACGAGATGGACACCTATGATCCGAATCAATTAAGAT
ATGAGAAATTCTTCTTTACAGTGAAAATGACGGTTAGATCTAATCGTCCGTTCAGAA
CATACTCAGATGTGGCAGCCGCTGTATCCCATTGGGATCACATGTACATCGGAATGG
CAGGGAAACGTCCCTTCTACAAAATCTTGGCTTTTTTGGGTTCTTCTAATCTAAAGGC
CACTCCAGCGGTATTGGCAGATCAAGGTCAACCAGAGTATCACGCTCACTGCGAAG
GCAGGGCTTATTTGCCACATAGGATGGGGAAGACCCCTCCCATGCTCAATGTACCAG
AGCACTTCAGAAGACCATTCAATATAGGTCTTTACAAGGGAACGATTGAGCTCACA
ATGACCATCTACGATGATGAGTCACTGGAAGCAGCTCCTATGATCTGGGATCATTTC
AATTCTTCCAAATTTTCTGATTTCAGAGAGAAGGCCTTAATGTTTGGCCTGATTGTCG
AGAAAAAGGCATCTGGAGCGTGGGTCCTGGATTCTATCAGCCACTTCAAATGAGCT
AGTCTAGCTTCCAGCTTCTGAACAATCCCCGGTTTACTCAGTCTCTCCTAATTCCAGC
CTTTCGAACAACTAATATCCTGTCTTTTCTATCCCTATGAAAAAAACTAACAGAGAT
CGATCTGTTTCCTTGACACCATGAAGTGCCTTTTGTACTTAGCTTTTTTATTCATCGG
GGTGAATTGCAAGTTCACCATAGTTTTTCCACACAACCGAAAAGGAAACTGGAAAA
ATGTTCCTTCCAATTACCATTATTGCCCGTCAAGCTCAGATTTAAATTGGCATAATGA
CTTAATAGGCACAGCCTTACAAGTCAAAATGCCCAAGAGTCACAAGGCTATTCAAG
CAGACGGTTGGATGTGTCATGCTTCCAAATGGGTCACTACTTGTGATTTCCGCTGGT
ACGGACCGGAGTATATAACACATTCCATCCGATCCTTCACTCCATCTGTAGAACAAT
GCAAGGAAAGCATTGAACAAACGAAACAAGGAACTTGGCTGAATCCAGGCTTCCCT
CCTCAAAGTTGTGGATATGCAACTGTGACGGATGCTGAAGCAGCGATTGTCCAGGT
GACTCCTCACCATGTGCTTGTTGATGAATACACAGGAGAATGGGTTGATTCACAGTT
CATCAACGGAAAATGCAGCAATGACATATGCCCCACTGTCCATAACTCCACAACCT
GGCATTCCGACTATAAGGTCAAAGGGCTATGTGATTCTAACCTCATTTCCATGGACA
TCACCTTCTTCTCAGAGGACGGAGAGCTATCATCCCTAGGAAAGGAGGGCACAGGG
TTCAGAAGTAACTACTTTGCTTATGAAACTGGAGACAAGGCCTGCAAAATGCAGTA
CTGCAAGCATTGGGGAGTCAGACTCCCATCAGGTGTCTGGTTCGAGATGGCTGATAA
GGATCTCTTTGCTGCAGCCAGATTCCCTGAATGCCCAGAAGGGTCAAGTATCTCTGC
TCCATCTCAGACCTCAGTGGATGTAAGTCTCATTCAGGACGTTGAGAGGATCTTGGA
TTATTCCCTCTGCCAAGAAACCTGGAGCAAAATCAGAGCGGGTCTTCCCATCTCTCC
AGTGGATCTCAGCTATCTTGCTCCTAAAAACCCAGGAACCGGTCCTGTCTTTACCAT
AATCAATGGTACCCTAAAATACTTTGAGACCAGATACATCAGAGTCGATATTGCTGC
TCCAATCCTCTCAAGAATGGTCGGAATGATCAGTGGAACTACCACAGAAAGGGAAC
TGTGGGATGACTGGGCTCCATATGAAGACGTGGAAATTGGACCCAATGGAGTTCTG
AGGACCAGTTCAGGATATAAGTTTCCTTTATATATGATTGGACATGGTATGTTGGAC
TCCGATCTTCATCTTAGCTCAAAGGCTCAGGTGTTTGAACATCCTCACATTCAAGAC
GCTGCTTCGCAGCTTCCTGATGATGAGACTTTATTTTTTGGTGATACTGGGCTATCCA
AAAATCCAATCGAGTTTGTAGAAGGTTGGTTCAGTAGTTGGAAGAGCTCTATTGCCT
CTTTTTGCTTTATCATAGGGTTAATCATTGGACTATTCTTGGTTCTCCGAGTTGGTAT
TTATCTTTGCATTAAATTAAAGCACACCAAGAAAAGACAGATTTATACAGACATAG
AGATGAACCGACTTGGAAAGTAACTCAAATCCTGCACAACAGATTCTTCATGTTTGA
ACCAAATCAACTTGTGATATCATGCTCAAAGAGGCCTTAATTATATTTTAATTTTTAA
TTTTTATGAAAAAAACTAACAGCAATCATGGAAGTCCACGATTTTGAGACCGACGA
GTTCAATGATTTCAATGAAGATGACTATGCCACAAGAGAATTCCTGAATCCCGATGA
GCGCATGACGTACTTGAATCATGCTGATTACAATTTGAATTCTCCTCTAATTAGTGAT
GATATTGACAATTTGATCAGGAAATTCAATTCTCTTCCGATTCCCTCGATGTGGGAT
AGTAAGAACTGGGATGGAGTTCTTGAGATGTTAACATCATGTCAAGCCAATCCCATC
TCAACATCTCAGATGCATAAATGGATGGGAAGTTGGTTAATGTCTGATAATCATGAT
GCCAGTCAAGGGTATAGTTTTTTACATGAAGTGGACAAAGAGGCAGAAATAACATT
TGACGTGGTGGAGACCTTCATCCGCGGCTGGGGCAACAAACCAATTGAATACATCA
AAAAGGAAAGATGGACTGACTCATTCAAAATTCTCGCTTATTTGTGTCAAAAGTTTT
TGGACTTACACAAGTTGACATTAATCTTAAATGCTGTCTCTGAGGTGGAATTGCTCA
ACTTGGCGAGGACTTTCAAAGGCAAAGTCAGAAGAAGTTCTCATGGAACGAACATA
TGCAGGATTAGGGTTCCCAGCTTGGGTCCTACTTTTATTTCAGAAGGATGGGCTTAC
TTCAAGAAACTTGATATTCTAATGGACCGAAACTTTCTGTTAATGGTCAAAGATGTG
ATTATAGGGAGGATGCAAACGGTGCTATCCATGGTATGTAGAATAGACAACCTGTT
CTCAGAGCAAGACATCTTCTCCCTTCTAAATATCTACAGAATTGGAGATAAAATTGT
GGAGAGGCAGGGAAATTTTTCTTATGACTTGATTAAAATGGTGGAACCGATATGCA
ACTTGAAGCTGATGAAATTAGCAAGAGAATCAAGGCCTTTAGTCCCACAATTCCCTC
ATTTTGAAAATCATATCAAGACTTCTGTTGATGAAGGGGCAAAAATTGACCGAGGT
ATAAGATTCCTCCATGATCAGATAATGAGTGTGAAAACAGTGGATCTCACACTGGTG
ATTTATGGATCGTTCAGACATTGGGGTCATCCTTTTATAGATTATTACACTGGACTAG
AAAAATTACATTCCCAAGTAACCATGAAGAAAGATATTGATGTGTCATATGCAAAA
GCACTTGCAAGTGATTTAGCTCGGATTGTTCTATTTCAACAGTTCAATGATCATAAA
AAGTGGTTCGTGAATGGAGACTTGCTCCCTCATGATCATCCCTTTAAAAGTCATGTT
AAAGAAAATACATGGCCCACAGCTGCTCAAGTTCAAGATTTTGGAGATAAATGGCA
TGAACTTCCGCTGATTAAATGTTTTGAAATACCCGACTTACTAGACCCATCGATAAT
ATACTCTGACAAAAGTCATTCAATGAATAGGTCAGAGGTGTTGAAACATGTCCGAA
TGAATCCGAACACTCCTATCCCTAGTAAAAAGGTGTTGCAGACTATGTTGGACACAA
AGGCTACCAATTGGAAAGAATTTCTTAAAGAGATTGATGAGAAGGGCTTAGATGAT
GATGATCTAATTATTGGTCTTAAAGGAAAGGAGAGGGAACTGAAGTTGGCAGGTAG
ATTTTTCTCCCTAATGTCTTGGAAATTGCGAGAATACTTTGTAATTACCGAATATTTG
ATAAAGACTCATTTCGTCCCTATGTTTAAAGGCCTGACAATGGCGGACGATCTAACT
GCAGTCATTAAAAAGATGTTAGATTCCTCATCCGGCCAAGGATTGAAGTCATATGAG
GCAATTTGCATAGCCAATCACATTGATTACGAAAAATGGAATAACCACCAAAGGAA
GTTATCAAACGGCCCAGTGTTCCGAGTTATGGGCCAGTTCTTAGGTTATCCATCCTT
AATCGAGAGAACTCATGAATTTTTTGAGAAAAGTCTTATATACTACAATGGAAGACC
AGACTTGATGCGTGTTCACAACAACACACTGATCAATTCAACCTCCCAACGAGTTTG
TTGGCAAGGACAAGAGGGTGGACTGGAAGGTCTACGGCAAAAAGGATGGAGTATC
CTCAATCTACTGGTTATTCAAAGAGAGGCTAAAATCAGAAACACTGCTGTCAAAGTC
TTGGCACAAGGTGATAATCAAGTTATTTGCACACAGTATAAAACGAAGAAATCGAG
AAACGTTGTAGAATTACAGGGTGCTCTCAATCAAATGGTTTCTAATAATGAGAAAAT
TATGACTGCAATCAAAATAGGGACAGGGAAGTTAGGACTTTTGATAAATGACGATG
AGACTATGCAATCTGCAGATTACTTGAATTATGGAAAAATACCGATTTTCCGTGGAG
TGATTAGAGGGTTAGAGACCAAGAGATGGTCACGAGTGACTTGTGTCACCAATGAC
CAAATACCCACTTGTGCTAATATAATGAGCTCAGTTTCCACAAATGCTCTCACCGTA
GCTCATTTTGCTGAGAACCCAATCAATGCCATGATACAGTACAATTATTTTGGGACA
TTTGCTAGACTCTTGTTGATGATGCATGATCCTGCTCTTCGTCAATCATTGTATGAAG
TTCAAGATAAGATACCGGGCTTGCACAGTTCTACTTTCAAATACGCCATGTTGTATT
TGGACCCTTCCATTGGAGGAGTGTCGGGCATGTCTTTGTCCAGGTTTTTGATTAGAG
CCTTCCCAGATCCCGTAACAGAAAGTCTCTCATTCTGGAGATTCATCCATGTACATG
CTCGAAGTGAGCATCTGAAGGAGATGAGTGCAGTATTTGGAAACCCCGAGATAGCC
AAGTTTCGAATAACTCACATAGACAAGCTAGTAGAAGATCCAACCTCTCTGAACATC
GCTATGGGAATGAGTCCAGCGAACTTGTTAAAGACTGAGGTTAAAAAATGCTTAAT
CGAATCAAGACAAACCATCAGGAACCAGGTGATTAAGGATGCAACCATATATTTGT
ATCATGAAGAGGATCGGCTCAGAAGTTTCTTATGGTCAATAAATCCTCTGTTCCCTA
GATTTTTAAGTGAATTCAAATCAGGCACTTTTTTGGGAGTCGCAGACGGGCTCATCA
GTCTATTTCAAAATTCTCGTACTATTCGGAACTCCTTTAAGAAAAAGTATCATAGGG
AATTGGATGATTTGATTGTGAGGAGTGAGGTATCCTCTTTGACACATTTAGGGAAAC
TTCATTTGAGAAGGGGATCATGTAAAATGTGGACATGTTCAGCTACTCATGCTGACA
CATTAAGATACAAATCCTGGGGCCGTACAGTTATTGGGACAACTGTACCCCATCCAT
TAGAAATGTTGGGTCCACAACATCGAAAAGAGACTCCTTGTGCACCATGTAACACA
TCAGGGTTCAATTATGTTTCTGTGCATTGTCCAGACGGGATCCATGACGTCTTTAGTT
CACGGGGACCATTGCCTGCTTATCTAGGGTCTAAAACATCTGAATCTACATCTATTT
TGCAGCCTTGGGAAAGGGAAAGCAAAGTCCCACTGATTAAAAGAGCTACACGTCTT
AGAGATGCTATCTCTTGGTTTGTTGAACCCGACTCTAAACTAGCAATGACTATACTT
TCTAACATCCACTCTTTAACAGGCGAAGAATGGACCAAAAGGCAGCATGGGTTCAA
AAGAACAGGGTCTGCCCTTCATAGGTTTTCGACATCTCGGATGAGCCATGGTGGGTT
CGCATCTCAGAGCACTGCAGCATTGACCAGGTTGATGGCAACTACAGACACCATGA
GGGATCTGGGAGATCAGAATTTCGACTTTTTATTCCAAGCAACGTTGCTCTATGCTC
AAATTACCACCACTGTTGCAAGAGACGGATGGATCACCAGTTGTACAGATCATTATC
ATATTGCCTGTAAGTCCTGTTTGAGACCCATAGAAGAGATCACCCTGGACTCAAGTA
TGGACTACACGCCCCCAGATGTATCCCATGTGCTGAAGACATGGAGGAATGGGGAA
GGTTCGTGGGGACAAGAGATAAAACAGATCTATCCTTTAGAAGGGAATTGGAAGAA
TTTAGCACCTGCTGAGCAATCCTATCAAGTCGGCAGATGTATAGGTTTTCTATATGG
AGACTTGGCGTATAGAAAATCTACTCATGCCGAGGACAGTTCTCTATTTCCTCTATC
TATACAAGGTCGTATTAGAGGTCGAGGTTTCTTAAAAGGGTTGCTAGACGGATTAAT
GAGAGCAAGTTGCTGCCAAGTAATACACCGGAGAAGTCTGGCTCATTTGAAGAGGC
CGGCCAACGCAGTGTACGGAGGTTTGATTTACTTGATTGATAAATTGAGTGTATCAC
CTCCATTCCTTTCTCTTACTAGATCAGGACCTATTAGAGACGAATTAGAAACGATTC
CCCACAAGATCCCAACCTCCTATCCGACAAGCAACCGTGATATGGGGGTGATTGTCA
GAAATTACTTCAAATACCAATGCCGTCTAATTGAAAAGGGAAAATACAGATCACAT
TATTCACAATTATGGTTATTCTCAGATGTCTTATCCATAGACTTCATTGGACCATTCT
CTATTTCCACCACCCTCTTGCAAATCCTATACAAGCCATTTTTATCTGGGAAAGATA
AGAATGAGTTGAGAGAGCTGGCAAATCTTTCTTCATTGCTAAGATCAGGAGAGGGG
TGGGAAGACATACATGTGAAATTCTTCACCAAGGACATATTATTGTGTCCAGAGGA
AATCAGACATGCTTGCAAGTTCGGGATTGCTAAGGATAATAATAAAGACATGAGCT
ATCCCCCTTGGGGAAGGGAATCCAGAGGGACAATTACAACAATCCCTGTTTATTATA
CGACCACCCCTTACCCAAAGATGCTAGAGATGCCTCCAAGAATCCAAAATCCCCTGC
TGTCCGGAATCAGGTTGGGCCAATTACCAACTGGCGCTCATTATAAAATTCGGAGTA
TATTACATGGAATGGGAATCCATTACAGGGACTTCTTGAGTTGTGGAGACGGCTCCG
GAGGGATGACTGCTGCATTACTACGAGAAAATGTGCATAGCAGAGGAATATTCAAT
AGTCTGTTAGAATTATCAGGGTCAGTCATGCGAGGCGCCTCTCCTGAGCCCCCCAGT
GCCCTAGAAACTTTAGGAGGAGATAAATCGAGATGTGTAAATGGTGAAACATGTTG
GGAATATCCATCTGACTTATGTGACCCAAGGACTTGGGACTATTTCCTCCGACTCAA
AGCAGGCTTGGGGCTTCAAATTGATTTAATTGTAATGGATATGGAAGTTCGGGATTC
TTCTACTAGCCTGAAAATTGAGACGAATGTTAGAAATTATGTGCACCGGATTTTGGA
TGAGCAAGGAGTTTTAATCTACAAGACTTATGGAACATATATTTGTGAGAGCGAAA
AGAATGCAGTAACAATCCTTGGTCCCATGTTCAAGACGGTCGACTTAGTTCAAACAG
AATTTAGTAGTTCTCAAACGTCTGAAGTATATATGGTATGTAAAGGTTTGAAGAAAT
TAATCGATGAACCCAATCCCGATTGGTCTTCCATCAATGAATCCTGGAAAAACCTGT
ACGCATTCCAGTCATCAGAACAGGAATTTGCCAGAGCAAAGAAGGTTAGTACATAC
TTTACCTTGACAGGTATTCCCTCCCAATTCATTCCTGATCCTTTTGTAAACATTGAGA
CTATGCTACAAATATTCGGAGTACCCACGGGTGTGTCTCATGCGGCTGCCTTAAAAT
CATCTGATAGACCTGCAGATTTATTGACCATTAGCCTTTTTTATATGGCGATTATATC
GTATTATAACATCAATCATATCAGAGTAGGACCGATACCTCCGAACCCCCCATCAGA
TGGAATTGCACAAAATGTGGGGATCGCTATAACTGGTATAAGCTTTTGGCTGAGTTT
GATGGAGAAAGACATTCCACTATATCAACAGTGTTTGGCAGTTATCCAGCAATCATT
TCCGATTAGGTGGGAGGCTATTTCAGTAAAAGGAGGATACAAGCAGAAGTGGAGTA
CTAGAGGTGATGGGCTCCCAAAAGATACCCGAATTTCAGACTCCTTGGCCCCAATCG
GGAACTGGATCAGATCTTTGGAATTGGTCCGAAACCAAGTTCGTCTAAATCCATTCA
ATAAGATCTTGTTCAATCAGCTATGTCGTACAGTGGATAATCATTTGAAGTGGTCAA
ATTTGCGAAAAAACACAGGAATGATTGAATGGATCAATGGGCGAATTTCAAAAGAA
GACCGGTCTATACTGATGTTGAAGAGTGACCTACATGAGGAAAACTCTTGGAGAGA
TTAAAAAATCAGGAGGAGACTCCAAACTTTAAGTATGAAAAAAACTTTGATCCTTA
AGACCCTCTTGTGGTTTTTATTTTTTTATCTGGTTTTGTGGTCTTCGT

1. A composition comprising one or more recombinant viral vectors and one or more respiratory syncytial virus (RSV) proteins.

2. The composition of claim 1, wherein the composition comprises at least two RSV proteins expressed in the same recombinant viral vector.

3. The composition of claim 1, wherein the one or more recombinant viral vector is recombinant vesicular stomatitis virus (rVSV).

4. The composition of claim 3, wherein the composition comprises two or more rVSV vectors.

5. The composition of claim 1, wherein the RSV protein is G protein.

6. The composition of claim 1, wherein the RSV protein is mG protein (codon-optimized, membrane bound G protein).

7. The composition of claim 1, wherein the RSV G protein is codon-optimized.

8. The composition of claim 7, wherein the codon-optimized RSV G protein is encoded by a nucleic acid comprising SEQ ID NO: 1.

9. The composition of claim 1, wherein the RSV protein is F protein.

10. The composition of claim 9, wherein the F-protein is codon-optimized F protein, pre-fusion conformation stabilized F-protein, or post-fusion F protein.

11. The composition of claim 1, wherein the RSV protein is tandem-expressed F and G proteins.

12. The composition of claim 1, wherein the RSV protein is M2-1 protein.

13. The composition of claim 1, wherein the RSV protein is N protein.

14. The composition of claim 2, further wherein RSV G protein is one of the RSV proteins.

15. The composition of claim 2, wherein mG protein is one of the RSV proteins.

16. The composition of claim 6, wherein the composition comprises at least one additional RSV protein selected from the group comprising F, M2-1, and N proteins.

17. The composition of claim 1, further comprising an adjuvant.

18. The composition of claim 17, wherein the adjuvant is Hsp70.

19. A vaccine comprising a composition of claim 1 in a carrier.

20. A method of eliciting an immune response against RSV in a subject, the method comprising administering to the subject a composition of claim 1 or the vaccine of claim 19.


SEQUENCE LISTING (22 sequences)

SEQ ID NO: 1 - DNA, 897 bp, Artificial Sequence
  synthetic construct
atgtccaaaaacaaggaccaacgcaccgctaagacattagaaaggacctgggacactctcaatcatttattattcatatcatcgtgcttatataagttaaatcttaaatctgtagcacaaatcacattatccattctggcaatgataatctcaacttcacttataattgcagccatcatattcatagcctcggcaaaccacaaagtcacaccaacaactgcaatcatacaagatgcaacaagccagatcaagaacacaaccccaacatacctcacccagaatcctcagcttggaatcagtccctctaatccgtctgaaattacatcacaaatcaccaccatactagcttcaacaacaccaggagtcaagtcaaccctgcaatccacaacagtcaagaccaaaaacacaacaacaactcaaacacaacccagcaagcccaccacaaaacaacgccaaaacaaaccaccaagcaaacccaataatgattttcactttgaagtgttcaactttgtaccctgcagcatatgcagcaacaatccaacctgctgggctatctgcaaaagaataccaaacaaaaaaccaggaaagaaaaccactaccaagcccacaaaaaaaccaaccctcaagacaaccaaaaaagatcccaaacctcaaaccactaaatcaaaggaagtacccaccaccaagcccacagaagagccaaccatcaacaccaccaaaacaaacatcataactacactactcacctccaacaccacaggaaatccagaactcacaagtcaaatggaaaccttccactcaacttcctccgaaggcaatccaagcccttctcaagtctctacaacatccgagtacccatcacaaccttcatctccacccaacacaccacgccagtag

SEQ ID NO: 2 - DNA, 897 bp, Artificial Sequence
  synthetic construct
atgagcaagaacaaggaccagcggaccgccaagaccctggagcggacctgggacaccctgaaccacctgctgttcatcagcagctgcctgtacaagctgaacctgaagagcgtggcccagatcaccctgagcatcctggccatgatcatcagcaccagcctgatcatcgccgccatcatcttcatcgccagcgccaaccacaaggtgacccccaccaccgccatcatccaggacgccaccagccagatcaagaacaccacccccacctacctgacccagaacccccagctgggcatcagccccagcaaccccagcgagatcaccagccagatcaccaccatcctggccagcaccacccccggcgtgaagagcaccctgcagagcaccaccgtgaagaccaagaacaccaccaccacccagacccagcccagcaagcccaccaccaagcagcggcagaacaagcctcccagcaagcccaacaacgacttccacttcgaggtgttcaacttcgtgccctgcagcatctgcagcaacaaccccacctgctgggccatctgcaagcggattcccaacaagaagcccggcaagaagaccaccaccaagcccaccaagaagcccaccctgaagaccaccaagaaggaccccaagccccagaccaccaagagcaaggaggtgcccaccaccaagcccaccgaggagcccaccatcaacaccaccaagaccaacatcatcaccaccctgctgaccagcaacaccaccggcaaccccgagctgaccagccagatggagaccttccacagcaccagcagcgagggcaaccccagccccagccaggtgagcaccaccagcgagtaccccagccagcccagcagccctcccaacacccctcggcagtag

SEQ ID NO: 3 - DNA, 897 bp, Artificial Sequence
  synthetic construct
atgagcaagaacaaggaccagcggaccgccaagaccctggagcggacctgggacaccctgaaccacctgctgttcatcagcagctgcctgtacaagctgaacctgaagagcgtggcccagatcaccctgagcatcctggccattatcatcagcaccagcctgatcatcgccgccatcatcttcatcgccagcgccaaccacaaggtgacccccaccaccgccatcatccaggacgccaccagccagatcaagaacaccacccccacctacctgacccagaacccccagctgggcatcagccccagcaaccccagcgagatcaccagccagatcaccaccatcctggccagcaccacccccggcgtgaagagcaccctgcagagcaccaccgtgaagaccaagaacaccaccaccacccagacccagcccagcaagcccaccaccaagcagcggcagaacaagcctcccagcaagcccaacaacgacttccacttcgaggtgttcaacttcgtgccctgcagcatctgcagcaacaaccccacctgctgggccatctgcaagcggattcccaacaagaagcccggcaagaagaccaccaccaagcccaccaagaagcccaccctgaagaccaccaagaaggaccccaagccccagaccaccaagagcaaggaggtgcccaccaccaagcccaccgaggagcccaccatcaacaccaccaagaccaacatcatcaccaccctgctgaccagcaacaccaccggcaaccccgagctgaccagccagatggagaccttccacagcaccagcagcgagggcaaccccagccccagccaggtgagcaccaccagcgagtaccccagccagcccagcagccctcccaacacccctcggcagtag

SEQ ID NO: 4 - DNA, 897 bp, Artificial Sequence
  synthetic construct
atgagcaagaacaaggaccagcggaccgccaagaccctggagcggacctgggacaccctgaaccacctgctgttcatcagcagctgcctgtacaagctgaacctgaagagcgtggcccagatcaccctgagcatcctggccatgatcatcagcaccagcctgatcatcgccgccatcatcttcatcgccagcgccaaccacaaggtgacccccaccaccgccatcatccaggacgccaccagccagatcaagaacaccacccccacctacctgacccagaacccccagctgggcatcagccccagcaaccccagcgagatcaccagccagatcaccaccatcctggccagcaccacccccggcgtgaagagcaccctgcagagcaccaccgtgaagaccaagaacaccaccaccacccagacccagcccagcaagcccaccaccaagcagcggcagaacaagcctcccagcaagcccaacaacgacttccacttcgaggtgttcaacttcgtgccctgcagcatctgcagcaacaaccccacctgctgggccatctccaagcggattcccaacaagaagcccggcaagaagaccaccaccaagcccaccaagaagcccaccctgaagaccaccaagaaggaccccaagccccagaccaccaagagcaaggaggtgcccaccaccaagcccaccgaggagcccaccatcaacaccaccaagaccaacatcatcaccaccctgctgaccagcaacaccaccggcaaccccgagctgaccagccagatggagaccttccacagcaccagcagcgagggcaaccccagccccagccaggtgagcaccaccagcgagtaccccagccagcccagcagccctcccaacacccctcggcagtag

SEQ ID NO: 5 - DNA, 756 bp, Artificial Sequence
  synthetic construct
atgatcatcagcaccagcctgatcatcgccgccatcatcttcatcgccagcgccaaccacaaggtgacccccaccaccgccatcatccaggacgccaccagccagatcaagaacaccacccccacctacctgacccagaacccccagctgggcatcagccccagcaaccccagcgagatcaccagccagatcaccaccatcctggccagcaccacccccggcgtgaagagcaccctgcagagcaccaccgtgaagaccaagaacaccaccaccacccagacccagcccagcaagcccaccaccaagcagcggcagaacaagcctcccagcaagcccaacaacgacttccacttcgaggtgttcaacttcgtgccctgcagcatctgcagcaacaaccccacctgctgggccatctgcaagcggattcccaacaagaagcccggcaagaagaccaccaccaagcccaccaagaagcccaccctgaagaccaccaagaaggaccccaagccccagaccaccaagagcaaggaggtgcccaccaccaagcccaccgaggagcccaccatcaacaccaccaagaccaacatcatcaccaccctgctgaccagcaacaccaccggcaaccccgagctgaccagccagatggagaccttccacagcaccagcagcgagggcaaccccagccccagccaggtgagcaccaccagcgagtaccccagccagcccagcagccctcccaacacccctcggcagtag

SEQ ID NO: 6 - DNA, 897 bp, Artificial Sequence
  synthetic construct
atgtctaaaaacaaggatcagcgaaccgccaaaaccctggagcgtacatgggatacactcaaccaccttctgttcatatctagctgcctttacaaacttaatctcaaaagcgtcgcccagattaccctctcaatactggctatgataatctccacctctttgataatagccgctatcattttcatagcttctgcaaaccacaaagtaactccaaccacagctataatacaagatgccacctctcagattaaaaataccacacccacatatcttactcagaatcctcaattgggaatcagcccatctaagccatccgagattacttcacagatcaccacaatactcgcatccacaacaccaggggtcaaatccaccctgcaatcaactaccgtgaaaactaaaaagaccactacaacacagactcaacccagcaagcctacaacaaagcaacgccagaataagccaccttctaagccaaacaatgatttccattttgaggtctttaatttcgtgccttgctctatatgttccaacaagccaacttgctgggccatttgcaaacgcatcccaaataagaaacccggtaagaaaaccacaaccaagccaactaaaaagccaactttgaagactaccaaaaaggaccctaagccccagacaactaaatcaaaagaagtcccaactactaagccaactgaggaaccaacaataaagactacaaaaaccaacatcatcacaacccttcttactagcaagactactggtaaccccgagctgacaagccagatggagacattccacagtacaagcagcgaaggaaacccaagccctagtcaagtgtccactacctcagaataccccagccagccttcctcacctcctaacacaccccggcaatag

SEQ ID NO: 7 - DNA, 921 bp, Artificial Sequence
  synthetic construct
cagcaatctcgagatgtctaaaaacaaggatcagcgaaccgccaaaaccctggagcgtacatgggatacactcaaccaccttctgttcatatctagctgcctttacaaacttaatctcaaaagcgtcgcccagattaccctctcaatactggctattataatctccacctctttgataatagccgctatcattttcatagcttctgcaaaccacaaagtaactccaaccacagctataatacaagatgccacctctcagattaaaaataccacacccacatatcttactcagaatcctcaattgggaatcagcccatctaagccatccgagattacttcacagatcaccacaatactcgcatccacaacaccaggggtcaaatccaccctgcaatcaactaccgtgaaaactaaaaagaccactacaacacagactcaacccagcaagcctacaacaaagcaacgccagaataagccaccttctaagccaaacaatgatttccattttgaggtctttaatttcgtgccttgctctatatgttccaacaagccaacttgctgggccatttgcaaacgcatcccaaataagaaacccggtaagaaaaccacaaccaagccaactaaaaagccaactttgaagactaccaaaaaggaccctaagccccagacaactaaatcaaaagaagtcccaactactaagccaactgaggaaccaacaataaagactacaaaaaccaacatcatcacaacccttcttactagcaagactactggtaaccccgagctgacaagccagatggagacattccacagtacaagcagcgaaggaaacccaagccctagtcaagtgtccactacctcagaataccccagccagccttcctcacctcctaacacaccccggcaatagcccgggttcat

SEQ ID NO: 8 - DNA, 84 bp, Artificial Sequence
  synthetic construct
ttccacttcgaggtgttcaacttcgtgccctgcagcatctgcagcaacaaccccacctgctgggccatctgcaagcggattccc

SEQ ID NO: 9 - DNA, 303 bp, Artificial Sequence
  synthetic construct
accgtgaagaccaagaacaccaccaccacccagacccagcccagcaagcccaccaccaagcagcggcagaacaagcctcccagcaagcccaacaacgacttccacttcgaggtgttcaacttcgtgccctgcagcatctgcagcaacaaccccacctgctgggccatctgcaagcggattcccaacaagaagcccggcaagaagaccaccaccaagcccaccaagaagcccaccctgaagaccaccaagaaggaccccaagccccagaccaccaagagcaaggaggtgcccaccaccaagccc

SEQ ID NO: 10 - DNA, 1725 bp, Artificial Sequence
  synthetic construct
atggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgttttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggcgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaa

SEQ ID NO: 11 - DNA, 1941 bp, Artificial Sequence
  synthetic construct
atggagctgctcatcctgaaggccaacgccatcaccaccatcctcaccgccgtgaccttctgcttcgccagcggccagaatatcacagaggaattttatcagtctacttgtagtgccgtcagtaaaggatatctgagcgctctcagaacaggatggtacactagtgttattacaatagaattgtcaaatatcaagaaaaataagtgcaacggtactgacgctaaggttaagctcatcaaacaggaacttgataaatataagaacgcagttacagaacttcagcttcttatgcagtccacacaagccaccaataataaagctaaaaaagaactcccacggttcatgaactacacactgaacaatgcaaaaaaaaccaacgtaacccttagcaagaaaaagaaaaaaaagttccttggcttcctcctcggagtaggcagcgctattgcaagtggggtagccgtgtgtaaggttttgcatctcgaaggagaagtgaataaaataaagagcgccttgctgtccactaataaggccgtagtcagccttagcaatggcgtatccgttctgacctttaaagtactggatttgaagaactacatcgataaacagcttctccccattttgaataagcaatcatgttctatcagtaacatagaaaccgtcatcgaattccaacaaaaaaacaatcggcttttggaaataactcgtgaattttctgtaaacgcaggcgtgacaactcccgtatcaacctacatgttgaccaatagcgaactgctgtcactcattaacgacatgccaatcactaacgaccagaaaaaacttatgagcaataatgtacagattgtaagacagcaaagttacagcataatgtgcattattaaggaagaagttttggcatacgttgtccaactccccctttatggggtcattgataccccctgctggaagctgcatactagcccattgtgtactaccaacaccaaagagggtagtaacatatgcctcaccagaactgaccgaggctggtactgtgataatgctggaagtgtcagtttctttcctcaagcagagacctgcaaagttcagtccaaccgcgtgttctgtgatacaatgaactccctgacactccctagcgaagtcaacctttgtaacgtcgatatatttaatcctaaatacgattgtaagatcatgacttcaaaaactgacgtatcctcttccgttattacttctttgggtgccatagttagttgctacggcaaaacaaaatgcaccgcatctaataaaaacagaggaattattaagacattttcaaatggttgcgactacgttagtaataaaggtgtagatacagtaagtgttggtaacaccctctattacgtgaacaaacaggaagggaaaagcctgtacgtgaaaggggagcccataatcaacttctacgacccccttgtatttcctagtgatgaatttgacgcctccatcagtcaagtgaacgaaaagatcaaccaaagccttgctttcatccgcaaatccgatgagttgctccacaatattaaaggctcgggatatataccggaggccccgcgagatggtcaagcttatgtgcgcaaagacggtgagtgggtcttgttatctacatttttgggtaacactaatagtggaggtagcacgacgacaattactaataataactcgggaactaactcaagctccactacctacactgtcaaatctggtgatacattgtggggcataagtcaaagatatggtatttcagtagcccaaattcaatcggcgaataatttaaagagcacaataatttacataggccagaagctcgtcctgacaggttccgcctcgtcaaccaatagcggaggcagcaacaacagtgcttcaacgacacccaccacctcggttactcctgctaagccaacaagtcaaacaact

SEQ ID NO: 12 - DNA, 1725 bp, Artificial Sequence
  synthetic construct
atggaacttcttatattgaaggcaaacgcaatcaccaccattttgactgcggttacattctgtttcgcctcaggtcaaaatattacagaagaattctaccagagcacatgctcagcggtatcaaagggttacttgtcagcccttaggaccggatggtatacctctgtaataacaatagaactttcaaacattaaaaaaaataagtgcaacgggaccgatgcaaaagttaaactgatcaagcaagaactggacaagtataaaaacgcagtcactgaacttcaacttcttatgcagtccacgcaagccactaataataaggctaagaaagaactgccaaggtttatgaactataccctgaacaacgcgaagaagactaatgtcacgttgtcaaaaaagaaaaagaaaaaattcctggggttcctgctcggagtaggcagtgcaatcgcgtctggagtagccgtatgtaaagtattgcaccttgaaggagaagtaaacaaaataaagagcgctctgctctctacgaacaaagctgttgtaagtctgagcaatggcgtctcagtcctgacatttaaagttcttgatttgaaaaattatattgacaaacaactcctccctatcctcaacaaacagtcttgctctatttcaaatattgagacagttatcgaatttcagcaaaaaaacaataggctccttgaaatcacacgagaattttctgtaaacgctggagtcacaacaccagtatctacgtatatgctcaccaattccgaacttctttcattgataaatgatatgcccataacaaacgaccagaaaaaattgatgtccaataatgtccaaatcgttcgccaacagagctattctatcatgtgtataataaaagaggaagttctcgcttacgttgtccaactgccgctgtacggggtgattgacacaccttgctggaaacttcatactagccctctgtgcacgactaacaccaaggaaggatcaaatatctgcctcacgcgaactgacaggggttggtactgtgataacgctggttccgtgtcattttttcctcaagctgagacgtgtaaagtacagtccaatcgagttttctgcgatactatgaactcactcaccttgccgtcagaggtgaacctctgtaacgtagatatatttaacccgaaatacgactgtaagattatgacttcaaagaccgatgtgtcaagctccgtcattacctccttgggagcaattgtttcttgctatggtaagacgaagtgcactgcgagcaacaagaatcgcggtatcatcaagacgttctccaacggatgcgattatgtaagtaacaagggagttgacacggtgagtgtagggaacacgttgtactatgtaaacaagcaggaggggaagtccttgtatgtcaagggcgaacctattatcaacttctacgacccattggtgttccctagtgacgagtttgatgctagtatttcccaggtcaacgagaagataaaccaaagtttggctttcattaggaagagcgatgagcttctccacaatgtgaacgccgggaagagtacgactaatattatgatcacaaccatcataatcgtcattatcgttattttgctctcactgattgcagtcggacttctgctgtactgcaaagctcgcagtaccccagtcacgctttccaaggaccaactttcaggcattaataacatcgcattttctaattaa

SEQ ID NO: 13 - DNA, 1509 bp, Artificial Sequence
  synthetic construct
atggaacttttgatactgaaggcgaacgccataacgacgatcctgacagctgtaactttttgcttcgcgagcggtcaaaacataaccgaggaattttatcagtcaacgtgctctgctgttagcaaaggatatctcagcgcactcaggacgggctggtacacgtcagtcataacgattgagctgtctaatatcaagaagaacaaatgcaacggaacggacgccaaagtcaagctcataaaacaagaattggacaagtacaagaatgctgtgacggagcttcagctcttgatgcagtccacccaagcgacgaataatagagcgaggagagagctcccaagatttatgaactatacactgaacaatgcaaagaagactaatgtgacccttagcaagaaaagaaaaagaagagcgattgcaagtggagtggctgtgtcaaaggtcctgcaccttgaaggtgaggtgaacaagattaaatccgcgctgctttctacgaacaaagctgtcgttagtttgtccaatggcgtttcagtgctcacttccaaggtattggatttgaagaattatattgacaaacagctccttccgattgttaataaacagagttgctcaatttctaacatcgaaactgtcatagagtttcagcagaagaacaatcggctcttggaaataacaagggagttttcagtcaacgccggggtaacaacacccgtgtccacatacatgctgacaaactccgagttgctctctcttatcaacgacatgccaattacaaacgaccagaagaaattgatgtccaacaacgtccaaatcgtacgacagcagtcttattccattatgagtattattaaggaagaggtattggcttatgtagtacaactccccttgtacggggtaatagacaccccctgttggaaactgcatacgagtcccctgtgtacaaccaatacgaaggagggctccaatatatgtttgacaagaactgaccgcggctggtactgtgataatgctggtagtgttagcttctttccacaagcggagacttgcaaggtacaatctaatcgggttttctgcgatacgatgaactctctgactctgccgagtgaggtcaacctgtgcaacgtggacatattcaatccgaagtacgattgtaaaattatgacatccaagacagatgtaagcagctctgttattacgtcactgggcgctattgtgtcatgctacggtaagactaaatgtaccgcatccaataaaaacagggggattattaaaaccttcagcaacggatgcgattatgtcagcaataagggcgtggataccgtatccgttggcaatactctctattacgtaaataaacaggaaggcaaatctctctatgttaaaggcgaacctataatcaatttttacgatccgcttgtattcccttccgatgaattcgatgcctctatctctcaagttaacgaaaaaatcaatcaatctctggcatttattaggaagtcagatgaactccta

SEQ ID NO: 14 - DNA, 1725 bp, Artificial Sequence
  synthetic construct
atggaattgctcattttgaaagctaatgctataacaacaatactcacggctgtaactttttgctttgcctctggtcaaaacataacggaagagttttatcagtcaacgtgttcagccgtatcaaaagggtatcttagcgcactgcgcactggatggtacacgtctgtgattaccattgaactcagtaatatcaaggaaaataaatgcaacggcactgatgcaaaagtcaagctcataaaacaggagcttgacaagtacaaaaatgcggttacagaactccagctccttatgcaatctaccccagcaaccaacaacaaagccaagaaggagctgcccaggtttatgaactatacacttaacaacgcgaagaaaaccaatgtcactctcagtaaaaagaaaaaaaagaagttcttggggttccttctcggtgttggaagcgccattgcaagcggtgtagcagtttgcaaagttctccaccttgagggggaggtgaacaaaattaaatctgccctcctctcaactaacaaagccgtcgtcagcttgagtaacggcgtaagcgtactcactttcaaagttctcgatctgaagaactatattgataaacagctgctcccaatactgaacaagcagtcatgcagcatcagcaacattgaaaccgtgatagagttccagcagaaaaataataggcttttggagataactcgggagttttcagtcaacgcgggtgtaacaacgccagtttccacgtatatgctgacaaacagtgagctcctgagcctgataaatgatatgccaatcacaaacgatcagaaaaaactcatgtccaataacgttcagatagtacggcaacagagttacagcataatgtgcataattaaagaggaggtcctggcttatgttgtccagcttccactgtacggggttatagataccccatgttggaagctccatacatctcccctgtgtactactaacaccaaggagggaagcaatatatgtttgactcgcactgacaggggttggtactgtgataatgccgggtccgtgagcttttttccgcaggctgaaacttgcaaggtgcaatctaaccgagtgttctgtgacactatgaattctctgactctcccgtcagaagtaaacttgtgtaatgtcgacatatttaaccctaaatacgattgtaagatcatgacaagcaaaacagacgtctcaagttctgtcataacaagcttgggcgcgattgtgtcctgttatggtaaaaccaaatgcacggcgtccaacaaaaataggggcattattaaaactttttccaacggctgtgattacgtctccaataaaggagtggatacggtctcagttgggaatactctgtactatgttaacaaacaagagggcaagtctctttatgtgaaaggggaaccgattataaacttttacgacccgcttgtgttcccgtccgatgagttcgatgcgagtatttcccaagtcaacgagaagataaaccagtccctcgcgtttatccgcaaaagtgacgagctccttcataacgttaatgctggtaagtccactacgaacatcatgatcacaacaattatcatagtcattattgttatactgcttagcctgatcgctgtagggttgctcttgtactgtaaagcgaggtctaccccagttacccttagtaaagaccaattgagtgggatcaacaacattgcgttttccaattga

SEQ ID NO: 15 - DNA, 714 bp, Artificial Sequence
  synthetic construct
caacttctgtcatccagcaaatacaccatccaacggagcacaggagatagtattgatactcctaattatgatgtgcagaaacacatcaataagttatgtggcatgttattaatcacagaagatgctaatcataaattcactgggttaataggtatgttatatgcgatgtctaggttaggaagagaagacaccataaaaatactcagagatgcgggatatcatgtaaaagcaaatggagtagatgtaacaacacatcgtcaagacattaatggaaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgactctcctgattgtgggatgataatattatgtatagcagcattagtaataactaaattagcagcaggggacagatctggtcttacagccgtgattaggagagctaataatgtcctaaaaaatgaaatgaaacgttacaaaggcttactacccaaggacatagccaacagcttctatgaagtgtttgaaaaacatccccactttatagatgtttttgttcattttggtatagcacaatcttctaccagaggtggcagtagagttgaagggatttttgcaggattgtttatgaatgcctatggtgca

SEQ ID NO: 16 - DNA, 762 bp, Artificial Sequence
  synthetic construct
caacttctgtcatccagcaaatacaccatccaacggagcacaggagatagtattgatactcctaattatgatgtgcagaaacacatcaataagttatgtggcatgttattaatcacagaagatgctaatcataaattcactgggttaataggtatgttatatgcgatgtctaggttaggaagagaagacaccataaaaatactcagagatgcgggatatcatgtaaaagcaaatggagtagatgtaacaacacatcgtcaagacattaatggaaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgactctcctgattgtgggatgataatattatgtatagcagcattagtaataactaaattagcagcaggggacagatctggtcttacagccgtgattaggagagctaataatgtcctaaaaaatgaaatgaaacgttacaaaggcttactacccaaggacatagccaacagcttctatgaagtgtttgaaaaacatccccactttatagatgtttttgttcattttggtatagcacaatcttctaccagaggtggcagtagagttgaagggatttttgcaggattgtttatgaatgcctatggtgcagggcaagtgatgttacggtggggagtcttagcaaaatcagttaaaaat

SEQ ID NO: 17 - DNA, 213 bp, Artificial Sequence
  synthetic construct
gcaggattctaccatatattgaacaacccaaaagcatcattattatctttgactcaatttcctcacttctccagtgtagtattaggcaatgctgctggcctaggcataatgggagagtacagaggtacaccgaggaatcaagatctatatgatgcagcaaaggcatatgctgaacaactcaaagaaaatggtgtgattaactacagtgtacta

SEQ ID NO: 18 - DNA, 114 bp, Artificial Sequence
  synthetic construct
tctaccagaggtggcagtagagttgaagggatttttgcaggattgtttatgaatgcctatggtgcagggcaagtgatgttacggtggggagtcttagcaaaatcagttaaaaat

SEQ ID NO: 19 - DNA, 585 bp, Artificial Sequence
  synthetic construct
atgtcacgaaggaatccttgcaaatttgaaattcgaggtcattgcttaaatggtaagaggtgtcattttagtcataattattttgaatggccaccccatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctga

SEQ ID NO: 20 - DNA, 1926 bp, Artificial Sequence
  synthetic construct
atggccaaagccgcggcagtcggcatcgacctgggcaccacctactcctgcgtgggggtgttccaacacggcaaggtggagatcatcgccaacgaccagggcaaccgcaccacccccagctacgtggccttcacggacaccgagcggctcatcggggatgcggccaagaaccaggtggcgctgaacccgcagaacaccgtgtttgacgcgaagcgcctgattggccgcaagttcggcgacccggtggtgcagtcggacatgaagcactggcctttccaggtgatcaacgacggagacaagcccaaggtgcaggtgagctacaagggggagaccaaggcattctaccccgaggagatctcgtccatggtgctgaccaagatgaaggagatcgccgaggcgtacctgggctacccggtgaccaacgcggtgatcaccgtgccggcctacttcaacgactcgcagcgccaggccaccaaggatgcgggtgtgatcgcggggctcaacgtgctgcggatcatcaacgagcccacggccgccgccatcgcctacggcctggacagaacgggcaagggggagcgcaacgtgctcatctttgacctgggcgggggcaccttcgacgtgtccatcctgacgatcgacgacggcatcttcgaggtgaaggccacggccggggacacccacctgggtggggaggactttgacaacaggctggtgaaccacttcgtggaggagttcaagagaaaacacaagaaggacatcagccagaacaagcgagccgtgaggcggctgcgcaccgcctgcgagagggccaagaggaccctgtcgtccagcacccaggccagcctggagatcgactccctgtttgagggcatcgacttctacacgtccatcaccagggcgaggttcgaggagctgtgctccgacctgttccgaagcaccctggagcccgtggagaaggctctgcgcgacgccaagctggacaaggcccagattcacgacctggtcctggtcgggggctccacccgcatccccaaggtgcagaagctgctgcaggacttcttcaacgggcgcgacctgaacaagagcatcaaccccgacgaggctgtggcctacggggcggcggtgcaggcggccatcctgatgggggacaagtccgagaacgtgcaggacctgctgctgctggacgtggctcccctgtcgctggggctggagacggccggaggcgtgatgactgccctgatcaagcgcaactccaccatccccaccaagcagacgcagatcttcaccacctactccgacaaccaacccggggtgctgatccaggtgtacgagggcgagagggccatgacgaaagacaacaatctgttggggcgcttcgagctgagcggcatccctccggcccccaggggcgtgccccagatcgaggtgaccttcgacatcgatgccaacggcatcctgaacgtcacggccacggacaagagcaccggcaaggccaacaagatcaccatcaccaacgacaagggccgcctgagcaaggaggagatcgagcgcatggtgcaggaggcggagaagtacaaagcggaggacgaggtgcagcgcgagagggtgtcagccaagaacgccctggagtcctacgccttcaacatgaagagcgccgtggaggatgaggggctcaagggcaagatcagcgaggcggacaagaagaaggtgctggacaagtgtcaagaggtcatctcgtggctggacgccaacaccttggccgagaaggacgagtttgagcacaagaggaaggagctggagcaggtgtgtaaccccatcatcagcggactgtaccagggtgccggtggtcccgggcctgggggcttcggggctcagggtcccaagggagggtctgggtcaggccccaccattgaggaggtagattag

SEQ ID NO: 21 - DNA, 2682 bp, Artificial Sequence
  synthetic construct
atgtccaaaaacaaggatcaacgaacggctaaaacactggaaagaacttgggatactcttaatcaccttcttttcatcagctcctgtttgtataagttgaacttgaaaagtgtagcacaaattaccttgtcaattctggctatgattatttccactagtttgatcattgctgcgattatatttattgcttctgcaaatcataaggtaaccccgactacagcgatcattcaggacgctacaagtcaaataaagaacaccacaccgacgtacttgacccagaatccccagcttggcatcagtccttctaacccttctgaaatcacctcccaaatcaccactatccttgcgtctaccacacctggagtaaagagtacattgcagtctactaccgttaagaccaagaacacaaccacaactcaaacgcagccatctaagccaactaccaaacagcggcaaaataaacctccatctaaaccgaataacgattttcactttgaagtattcaactttgttccctgctcaatttgcagcaataatccgacctgctgggctatatgtaagcggataccaaataaaaagccaggaaagaaaactacaacaaaacctacgaagaagcctacactgaagaccacaaaaaaagacccaaaaccccagacaaccaagtccaaggaagttcccactactaagcccactgaagagcctaccataaataccaccaagacaaacatcataaccaccttgctcacctctaatactaccggaaaccctgagctcacttcccaaatggaaacgttccattcaactagtagtgagggcaacccgagtcccagccaggtctctacaacctcagaatacccctcccaacctagttcacccccaaatactccacggcagggatccggagagggaagaggaagtttgctgacatgtggagatgtggaggaaaatcccggtccaatggagcttctgatcctgaaagctaacgctattactactatacttaccgccgtaacattctgcttcgcctccggacaaaacatcacagaagagttctatcaatccacgtgcagcgctgtgtctaagggctatctgagcgcattgagaacggggtggtatacttccgtaattactatagagctgtcaaacattaagaaaaacaagtgtaacggtaccgacgctaaagtaaagctcatcaagcaggagctggataaatacaaaaatgctgtcactgaactccagcttcttatgcaatctacccaagcaaccaacaaccgggctaggcgcgaattgcccaggttcatgaattatacattgaacaacgccaaaaagactaatgtaaccctcagcaagaaacgcaagaggcggttcctgggatttcttctcggagtaggttccgctatagcgtccggagtagcggtctcaaaagtattgcatctggaaggcgaagttaacaaaattaagagcgcgctcctcagcaccaacaaggcggtagtcagcctcagcaacggcgtatctgttctcacatctaaagttttggacctgaaaaactatatagacaagcagttgcttccgatagtaaataagcaatcatgttccatttcaaacatagaaacggttatcgagtttcaacagaaaaataatagattgcttgagatcacaagagagttctctgtcaatgcaggtgtgactacgccggtcagcacatatatgctcacgaatagtgaactgctgagtcttataaatgatatgccgattactaatgaccaaaaaaagctcatgagcaacaatgtccaaatcgttcgacaacaaagttactctatcatgagcatcatcaaagaggaggttctcgcatatgtcgtgcagcttccgttgtatggtgtaatagataccccgtgctggaagctgcacacctctccactgtgcacaaccaatactaaagaggggtctaatatctgtctcacgagaacggatcgaggatggtactgcgataacgccggtagtgtgagcttcttcccccaggctgaaacctgtaaggtacagagtaacagggtattctgtgacactatgaactcactcacactgccaagtgaagtgaacctttgtaacgttgacatatttaatcccaagtacgactgcaaaatcatgacaagcaaaaccgacgtttcctcaagcgtcataacgagtttgggtgctatagtaagttgctatgggaaaaccaagtgcacggcatccaataagaacagagggatcataaaaacgttctccaacggatgtgactatgtgtcaaacaagggggttgatacggtatcagttggaaataccctttattatgtcaacaagcaggaaggaaagagcctctatgtaaaaggcgaacccataatcaatttttatgacccactcgtattccctagtgatgagttcgatgcctctattagccaggtaaatgagaagatcaaccagagtttggcctttatccgcaaatctgacgagctgctccataatgtcaatgcagggaaaagtacgactaatatcatgattactacgattattatcgtcatcatcgtcatcctcttgagtcttatagcggtagggctcctgctctactgtaaagcgcgctctacccctgtgacgctgtccaaagatcaactttctggcataaacaacattgcctttagtaattaa

SEQ ID NO: 22 - DNA, 11162 bp, Artificial Sequence
  synthetic construct
acgaagacaaacaaaccattattatcattaaaaggctcaggagaaactttaacagtaatcaaaatgtctgttacagtcaagagaatcattgacaacacagtcatagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctactctcgtctagatcaggcggtaggagagatagatgagatcgaagcacaacgagctgaaaagtccaattatgagttgttccaagaggacggagtggaagagcatactaggccctcttattttcaggcagcagatgattctgacacagaatctgaaccagaaattgaagacaatcaaggcttgtatgtaccagatccggaagctgagcaagttgaaggctttatacaggggcctttagatgactatgcagatgaggacgtggatgttgtattcacttcggactggaaacagcctgagcttgaatccgacgagcatggaaagaccttacggttgacattgccagagggtttaagtggagagcagaaatcccagtggcttttgacgattaaagcagtcgttcaaagtgccaaacactggaatctggcagagtgcacatttgaagcatcgggagaaggggtcatcataaaaaagcgccagataactccggatgtatataaggtcactccagtgatgaacacacatccgtaccaatcagaagccgtatcagatgtttggtctctctcaaagacatccatgactttccaacccaagaaagcaagtcttcagcctctcaccatatccttggatgaattgttctcatctagaggagaattcatctctgtcggaggtaacggacgaatgtctcataaagaggccatcctgctcggtctgaggtacaaaaagttgtacaatcaggcgagagtcaaatattctctgtagactatgaaaaaaagtaacagatatcacaatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggattctatcagccacttcaaatgagctagtctagcttccagcttctgaacaatccccggtttactcagtctctcctaattccagcctttcgaacaactaatatcctgtcttttctatccctatgaaaaaaactaacagagatcgatctgtttccttgacaccatgaagtgccttttgtacttagcttttttattcatcggggtgaattgcaagttcaccatagtttttccacacaaccgaaaaggaaactggaaaaatgttccttccaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtacggaccggagtatataacacattccatccgatccttcactccatctgtagaacaatgcaaggaaagcattgaacaaacgaaacaaggaacttggctgaatccaggcttccctcctcaaagttgtggatatgcaactgtgacggatgctgaagcagcgattgtccaggtgactcctcaccatgtgcttgttgatgaatacacaggagaatgggttgattcacagttcatcaacggaaaatgcagcaatgacatatgccccactgtccataactccacaacctggcattccgactataaggtcaaagggctatgtgattctaacctcatttccatggacatcaccttcttctcagaggacggagagctatcatccctaggaaaggagggcacagggttcagaagtaactactttgcttatgaaactggagacaaggcctgcaaaatgcagtactgcaagcattggggagtcagactcccatcaggtgtctggttcgagatggctgataaggatctctttgctgcagccagattccctgaatgcccagaagggtcaagtatctctgctccatctcagacctcagtggatgtaagtctcattcaggacgttgagaggatcttggattattccctctgccaagaaacctggagcaaaatcagagcgggtcttcccatctctccagtggatctcagctatcttgctcctaaaaacccaggaaccggtcctgtctttaccataatcaatggtaccctaaaatactttgagaccagatacatcagagtcgatattgctgctccaatcctctcaagaatggtcggaatgatcagtggaactaccacagaaagggaactgtgggatgactgggctccatatgaagacgtggaaattggacccaatggagttctgaggaccagttcaggatataagtttcctttatatatgattggacatggtatgttggactccgatcttcatcttagctcaaaggctcaggtgtttgaacatcctcacattcaagacgctgcttcgcagcttcctgatgatgagactttattttttggtgatactgggctatccaaaaatccaatcgagtttgtagaaggttggttcagtagttggaagagctctattgcctctttttgctttatcatagggttaatcattggactattcttggttctccgagttggtatttatctttgcattaaattaaagcacaccaagaaaagacagatttatacagacatagagatgaaccgacttggaaagtaactcaaatcctgcacaacagattcttcatgtttgaaccaaatcaacttgtgatatcatgctcaaagaggccttaattatattttaatttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaatttgaattctcctctaattagtgatgatattgacaatttgatcaggaaattcaattctcttccgattccctcgatgtgggatagtaagaactgggatggagttcttgagatgttaacatcatgtcaagccaatcccatctcaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggagtatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttggcagttatccagcaatcatttccgattaggtgggaggctatttcagtaaaaggaggatacaagcagaagtggagtactagaggtgatgggctcccaaaagatacccgaatttcagactccttggccccaatcgggaactggatcagatctttggaattggtccgaaaccaagttcgtctaaatccattcaataagatcttgttcaatcagctatgtcgtacagtggataatcatttgaagtggtcaaatttgcgaaaaaacacaggaatgattgaatggatcaatgggcgaatttcaaaagaagaccggtctatactgatgttgaagagtgacctacatgaggaaaactcttggagagattaaaaaatcaggaggagactccaaactttaagtatgaaaaaaactttgatccttaagaccctcttgtggtttttatttttttatctggttttgtggtcttcgt